(19) United States
(12) Patent Application Publication
(10) Pub. No.: US 2011/0251289 A1
(43) Pub. Date: Oct. 13, 2011

(54) NEW COMPOUNDS FOR THE TREATMENT OF CANCER

(75) Inventors: Moira Bode, Midrand (ZA); Robert Vleggaar, Pretoria (ZA); Paul J. Gates, Bristol (GB); R. Anitra Schultz, Pretoria (ZA); Dharmaral Naicker, Pretoria (ZA); Nel Fourie, Pretoria North (ZA)

(73) Assignees: CSIR, Pretoria (ZA); AGRICULTURAL RESEARCH COUNCIL, Pretoria (ZA); UNIVERSITY OF PRETORIA, Pretoria (ZA)

(21) Appl. No.: 12/996,091

(22) PCT Filed: Jun. 3, 2009
(86) PCT No.: PCT/IB2009/052348

§ 371 Date: Jun. 6, 2011

(30) Foreign Application Priority Data
Jun. 5, 2008 (ZA) 2008/04913

(51) Int. Cl. A61K 31/133; C07C 215/18; A61P 35/00; C07C 247/04; C07C 309/00; C07C 213/00; C07D 407/12

(52) U.S. Cl. 514/669; 564/507; 549/370; 552/11; 562/37

(57) ABSTRACT

The invention provides a method of preparing the stereoisomers of 1,11-diamino-6-aza-undecane-2,4,8,10-tetraol.

THIS invention relates to the treatment of cancer. In particular it relates to new compounds and compositions for the treatment of cancer and to methods of synthesising the compounds.

Gousiekte, which can be literally translated as “quick” disease, is one of the six most important plant toxicoses of livestock in South Africa. It is a plant-induced cardiomyopathy of domestic ruminants which is characterized by the sudden death of animals within a period of three to six weeks after the initial ingestion of toxic plant material. The six species of the three genera of the Rubiaceae family viz. Pachystigma pygmaeum, P. thamnus, and P. latifolium; Pavetta harborii and P. schumanniana, and Fadogia homblei have been identified as the causative agents of the disease. The disease was first identified in 1908 but because of the irregularity of the outbreaks, investigation of the disease was not pursued until a severe outbreak in 1915 was reported in which 1047 out of a flock of 1761 sheep died. Gousiekte is the last of the major plant poisonings in southern Africa to be investigated. The causal toxin has been isolated from Pachystigma pygmaeum, Pavetta harborii, P. schumanniana and Fadogia homblei but has hitherto not been identified.

The causal toxin of gousiekte has now been identified as the novel compound pavettamine (1) which is the 2S,4R,8R,10S stereoisomer of 1,11-diamino-6-aza-undecane-2,4,8,10-tetraol.

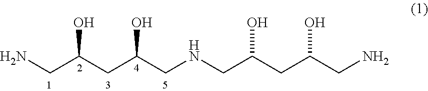

Pavettamine has been found to be an anticancer agent and an inhibitor of the growth of eukaryotic cells, without inhibiting the growth of prokaryotic cells. The structural elucidation of pavettamine was carried out by a combination of mass spectrometry and ¹³C NMR of pavettamine and its derivatives.

Electrospray ionization mass spectrometry (ESI-MS) established the molecular mass of pavettamine as 251 and the molecular formula as C₁₀H₂₅N₃O₄ by accurate mass determination of the [M+H]⁺, [M+Na]⁺ and [2M+Na]⁺ ions as well as the fragment ions formed from the [M+H]⁺ ion in an MS-MS analysis. The ¹³C NMR spectrum showed only 5 signals for the proton-bearing carbon atoms (see Table 1) and the ¹H NMR spectrum multiplet signals for only 8 protons. It is evident from the NMR data that the pavettamine molecule contains a symmetry element: which is either a C₂ axis or a symmetry plane. The multiplicities of the different ¹³C resonances were deduced from the proton-decoupled CH and CH₂ subspectra obtained using the DEPT pulse sequence. The signals of the proton-bearing carbon atoms were correlated with specific proton resonances in a two-dimensional (2-D) ¹³C{¹H} heteronuclear chemical shift correlation experiment (HETCOR) utilizing the one-bond (¹³C,¹H) spin-spin couplings. The assignments of the signals in the ¹H NMR spectrum were based on first-order analysis of the spin systems and chemical shift considerations and were confirmed by a two-dimensional (2D) (¹H,¹H) homonuclear chemical shift correlation (COSY) experiment and ¹H{¹H} spin-decoupling experiments. The 1,11-diamino-6-aza-undecane-2,4,8,10-tetraol structure was assigned to pavettamine on the basis of the above data.

The 1,11-diamino-6-aza-undecane-2,4,8,10-tetraol structure is in agreement with the fragmentation pattern (Scheme 1) derived from the analysis of the MS-MS spectrum of pavettamine as can be seen in Table 2.

TABLE 1
NMR Data for pavettamine (1) (in D₂O)

| δ_{H} | | δ_{C} | |
|---|---|---|---|
| H-1a | 2.851 (dd, J_{1a,1b} 13.2, J_{1a,2} 9.5) | C(1) | 46.65 T (J 143) |
| H-1b | 3.058 (dd, J_{1a,1b} 13.0, J_{1b,2} 3.0 | | |
| H-2 | 3.952 (m, J_{1a,2} 9.4, J_{1b,2} 3.1, J_{2,3} 6.5 | C(2) | 67.18 D (J 145) |
| H-3 | 1.679 (m) | C(3) | 40.97 T (J 127) |
| H-4 | 4.057 (m, J_{4,5a} 10.0, J_{4,5b} 2.8, J_{3,4} 6.3) | C(4) | 66.27 D (J 144) |
| H-5a | 3.000 (dd, J_{5a,5b} 13.0, J_{4,5a} 10.0) | C(5) | 54.56 T (J 144) |
| H-5b | 3.143 (dd, J_{5a,5b} 13.0, J_{4,5b} 2.9) | | |

TABLE 2
MS-MS of Pavettamine (1)

| Observed | | | Theoretical | | Match |
|---|---|---|---|---|---|
| Mass (m/z) | Identity | Formula | Mass | DBE | (ppm) |
| 252.191233 | [M + H]⁺ | C₁₀H₂₆N₃O₄ | 252.191769 | 0 | −2.13 |
| 235.165592 | [M − NH₃]⁺ | C₁₀H₂₃N₂O₄ | 235.165221 | 1 | +1.58 |
| 234.181234 | [M − H₂O]⁺ | C₁₀H₂₄N₃O₄ | 234.181206 | 1 | +0.12 |
| 217.154755 | | C₁₀H₂₁N₂O₃ | 217.154758 | 2 | +0.45 |
| 135.112484 | | C₅H₁₅N₂O₂ | 135.112796 | 0 | −2.31 |
| 118.085967 | | C₅H₁₂NO₂ | 118.086248 | 1 | −2.39 |
| 117.102321 | | C₅H₁₃N₂O | 117.102233 | 1 | +0.75 |
| 100.075477 | | C₅H₁₀NO | 100.075685 | 2 | −2.09 |
| 83.049182 | | C₅H₇O | 83.049137 | 3 | +0.53 |
| 82.064992 | | C₅H₈N | 82.065122 | 3 | −1.59 |

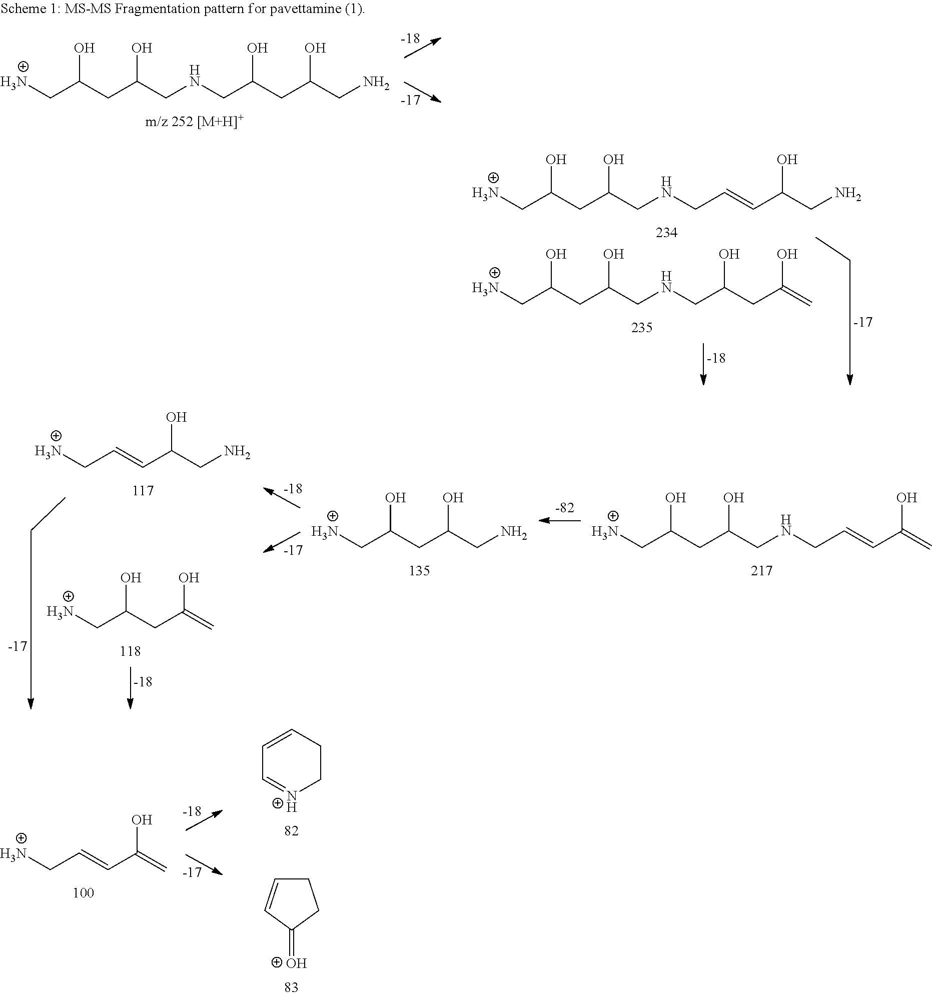

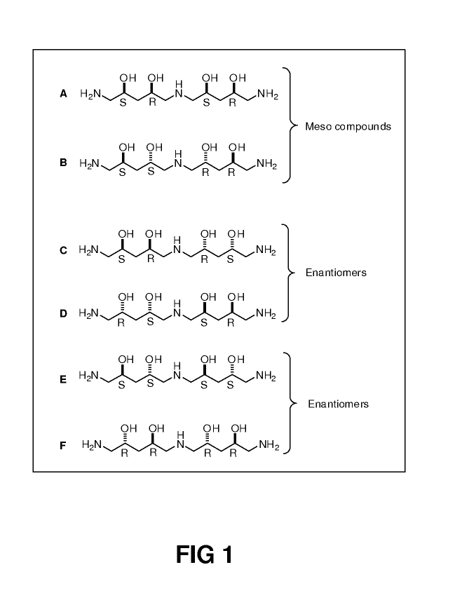

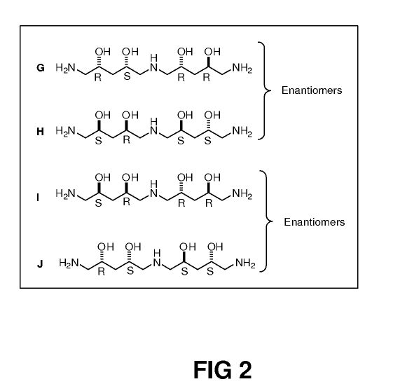

There are ten stereoisomers of the compound 1,11-diamino-6-aza-undecane-2,4,8,10-tetraol. Two of the stereoisomers are meso compounds, which are optically inactive, and eight of the stereoisomers are in the form of four pairs of enantiomers. As shown in FIG. 1, six of the stereoisomers (A, B, C, D, E and F) meet the symmetry criteria of pavettamine whereas four stereoisomers (G, H, I, and J) (FIG. 2) lack symmetry and can be excluded. A and B each have a plane of symmetry and are meso compounds, and C, D, E and F have C₂ symmetry axes and form two enantiomeric pairs C,D and E,F. Differentiation between the two groups of stereoisomers was possible by determining whether pavettamine showed optical activity. Since meso compounds are optically inactive, the presence of a C₂ symmetry element in pavettamine was established by the fact that the compound was optically active and showed a specific rotation of −19.5. Although the magnitude of the rotation remained in doubt as a result of solvent retained in the natural toxin obtained from the isolation procedure, the optical activity excluded the presence of a symmetry plane and thus the two possible meso stereoisomers for pavettamine.

The relative stereochemistry of pavettamine was established by ¹³C NMR analysis of the acetonide derivative of the 1,3-diol system present in the compound, a method developed by Rychnovsky. The amino groups present in pavettamine were first protected by converting the compound to the tri-Boc derivative by treatment with Boc₂O and Na₂CO₃ in aqueous dioxane (Scheme 2). The 1,3-diol system was then protected as the acetonide by acid-catalysed (TsOH) transacetalisation with 2,2-dimethoxypropane. The signals at δ_{C} 30.00Q, 19.87Q and 19.71Q for the 2,2-dimethyl groups of the formed dioxane rings as well as the signal at δ_{C} 98.73S for the acetal carbon atom established the syn stereochemistry of pavettamine. The absolute configuration as shown in (1) i.e. (2S,4R,8R,10S) (or ent-1) was therefore assigned to pavettamine.

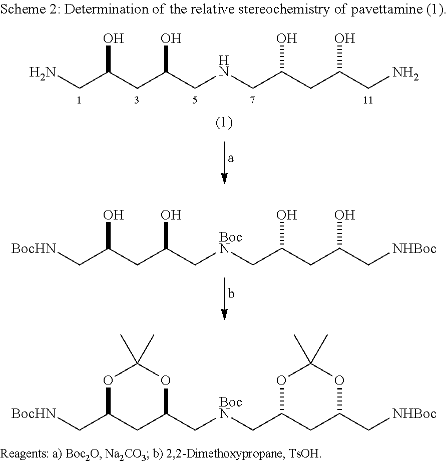

According to a first aspect of the invention there is provided the compound 1,11-diamino-6-aza-undecane-2,4,8,10-tetraol and its stereoisomers.

In particular, the invention provides the meso compounds

2S,4R,8S,10R (A) and 2S,4S,6R,10R (B).

as shown in FIG. 1, which are optically inactive, and the enantiomeric pairs

2S,4R,8R,10S (C) and 2R,4S,8S,10R (D)

2S,4S,8S,10S (E) and 2R,4R,8R,10R (F)

2R,4S,8R,10R (G) and 2S,4R,8S,10S (H) and

2S,4R,8R,10R (I) and 2R,4S,8S,10S (J).

as shown in FIGS. 1 and 2, which are optically active.

The thus provides a compound which is selected from the stereoisomers

2S,4R,8S,10R (A) and 2S,4S,6R,10R (B).

2S,4R,8R,10S (C) and 2R,4S,8S,10R (D)

2S,4S,8S,10S (E) and 2R,4R,8R,10R (F)

2R,4S,8R,10R (G) and 2S,4R,8S,10S (H)

2S,4R,8R,10R (I) and 2R,4S,8S,10S (J)

of 1,11-diamino-6-aza-undecane-2,4,8,10-tetraol.

The invention thus provides a compound which is selected from the stereoisomers

2S,4R,8S,10R (A) and 2S,4S,6R,10R (B).

2S,4R,8R,10S (C) and 2R,4S,8S,10R (D)

2S,4S,8S,10S (E) and 2R,4R,8R,10R (F)

2R,4S,8R,10R (G) and 2S,4R,8S,10S (H)

2S,4R,8R,10R (I) and 2R,4S,8S,10S (J)

of 1,11-diamino-6-aza-undecane-2,4,8,10-tetraol.

More particularly, the invention provides the stereoisomer (C) (pavettamine) which is (2S,4R,8R,10S)-1,11-diamino-6-aza-undecane-2,4,8,10-tetraol.

According to a second aspect of the invention, there is provided a method of selectively preparing a stereoisomer of 1,11-diamino-6-aza-undecane-2,4,8,10-tetraol, the method including the steps of

providing a 2,4-hydroxy protected 1,5-difunctionalised-2,4-hydroxypentane in which
the stereochemistry on the 2 and 4 positions is selected from one of 2R,4R; 2R,4S; 2S,4R and 2S,4S,
the functional groups on the 1 and 5 carbon atoms are selected so that the 1-carbon and the 5-carbon can both, selectively, be converted to a carboxylic acid or an aldehyde, and so that the functional groups on the 1-carbon and the 5-carbon can both, selectively, be converted to an amine,

converting either the 1-carbon or the 5-carbon into a carboxylic acid or an aldehyde to produce a first intermediate,

providing the same 2,4-hydroxy protected 1,5-difunctionalised-2,4-hydroxypentane and converting the functional group on the 1-carbon or the 5-carbon to an amine to produce a second intermediate,

condensing the first and the second intermediates to form the corresponding amide or imine; and

reducing the amide or the imine to produce the stereoisomer of 2,4,8,10-hydroxy protected 1,11-difunctionalised-6-aza-undecane-2,4,8,10-tetraol.

The stereoisomer may be selected from the stereoisomers

2S,4R,8S,10R (A) and 2S,4S,6R,10R (B).

2S,4R,8R,10S (C) and 2R,4S,8S,10R (D)

2S,4S,8S,10S (E) and 2R,4R,8R,10R (F)

2R,4S,8R,10R (G) and 2S,4R,8S,10S (H)

2S,4R,8R,10R (I) and 2R,4S,8S,10S (J).

The stereoisomer may be (2S,4R,8R,10S)-1,11-diamino-6-aza-undecane-2,4,8,10-tetraol.

The 1-substituent may be a hydroxyl group and the 5-substituent may be a hydroxyl group protected by a hydroxy protecting group. For example, the 5-substituent may be an O-trityl group. Separately converting the 1-hydroxy to a carboxylic acid or to an amine may be, respectively, by oxidation of the hydroxy group to a carboxylic acid or by conversion of the 1-hydroxy to a leaving group and converting the leaving group to an amine.

The functional group which is selected so that either the 1-carbon or the 5-carbon can be converted into an amine may be a protected hydroxyl group. The protected hydroxide group may be an O-tosyl group.

Where the 1-hydroxy group is an O-trityl group, the amide will be a 2,4,8,10-hydroxy protected 1,11-di-O,O-trityl-6-aza-5-oxo-undecane-2,4,8,10-tetraol. Reduction, followed by stepwise conversion of the O-trityl groups to amino groups, protection and deprotection of the secondary amino group and deprotection of the hydroxy protecting groups will then produce the desired stereoisomer of 1,11-diamino-6-aza-undecane-2,4,8,10-tetraol.

The functional group which is selected so that either the 1-carbon or the 5-carbon can be converted into a carboxylic acid or an aldehyde may be a hydroxyl group.

According to a third aspect of the invention, there is a provided a method of selectively preparing a stereoisomer of 11-diamino-6-aza-undecane-2,4,8,10-tetraol, which includes the steps of preparing a chiral precursor by

providing a 3,4-hydroxy-protected 3,4-dihydroxybutanoate ester selected from 3,4-dihydroxybutanoate esters having 3S or 3R stereochemistry,

introducing an additional carbon atom as a methylene group into the selected 3,4-dihydroxy protected 3,4-dihydroxybutanoate ester with an (R)-(+)-methylarylsulfoxide to produce a 4,5-dihydroxy-protected 1-(arylsulfinyl)-4,5-dihydroxypentan-2-one,

reducing the 4,5-hydroxy-protected 1-(arylsulfinyl)-4,5-dihydroxypentan-2-one with an alkylaluminium hydride to produce a 4,5-hydroxy-protected 1-(arylsulfinyl)-pentane-2,4,5-triol, in which the 4S isomer, in the presence of a zinc halide, produces the 2R,4S isomer and, in the absence of a zinc halide, produces the 2S,4S isomer, and the 4R isomer, in the presence of a zinc halide, produces the 2S,4R isomer, and in the absence of a zinc halide, produces the 2R,4R isomer,

deprotecting the 4,5-hydroxy-protected 1-(arylsulfinyl)-pentane-2,4,5-triol to produce the corresponding 1-(arylsulfinyl)-pentane-2,4,5-triol,

selectively protecting the 5-hydroxy group of the 1-(arylsulfinyl)-pentane-2,4,5-triol to produce a 5-hydroxy-protected 1-(arylsulfinyl)-pentane-2,4,5-triol,

selectively protecting the 2,4-hydroxy groups of the 5-hydroxy-protected 1-(arylsulfinyl)-pentane-2,4,5-triol to produce a 2,4,5-hydroxy-protected 1-(arylsulfinyl)-pentane-2,4,5-triol,

converting the 2,4,5-hydroxy-protected 1-(arylsulfinyl)-pentane-2,4,5-triol to the corresponding 2,4,5-hydroxy-protected 1-acetoxy-1-arylthio-5-pentane-2,4,5-triol by Pummerer rearrangement, and

reducing the 2,4,5-hydroxy-protected 1-acetoxy-1-arylthio-5-pentane-2,4,5-triol to produce the 2R,4S or 2S,4R or the 2S,4S or 2R,4R 1,2,4-hydroxy protected-pentane-1,2,4,5-tetraol, chiral precursor.

The 3,4-hydroxy-protected alkyl 3,4-dihydroxybutanoate ester having 3S or 3R stereochemistry may be prepared from (2R)-malic acid or (2S)-malic acid by esterification to produce the diester followed by selective reduction to produce an alkyl 3,4-dihydroxybutanoate in which the stereochemistry at position 3 will be R or S depending on whether (2R)-malic acid or (2S)-malic acid is selected as the starting material.

Preferably, the diester will be a diethyl ester. The selective reducing agent may be a borohydride in the presence of borane-methyl sulfide complex (BMS).

The (R)-(+)-methyl-p-aryl sulfoxide will preferably be the (R)-(+)-methyl p-tolylsulfoxide. This chiral compound can be prepared from p-toluenesulfinyl chloride by reaction with menthol to produce the menthyl ester followed by alkylation using methyl magnesium iodide in ether according to known procedures.

The reduction of the 4,5-dihydroxy-protected 1-(arylsulfinyl)-4,5-dihydroxypentan-2-one, is preferably carried out using diisobutyl aluminium hydride (DIBALH) and the zinc halide may be zinc chloride or zinc bromide. The selectivity of the reduction in the presence or in the absence of the zinc salt is an important feature of the invention in that it selectively provides the stereochemistry on carbon 2. Accordingly, by selecting and by carrying out the reduction step in the presence or in the absence of the zinc salt, the stereochemistry on carbon atoms 2 and 4 can selectively be controlled.

According to another aspect of the invention there is provided a method of selectively preparing a stereoisomer of 11-diamino-6-aza-undecane-2,4,8,10-tetraol, which includes the steps of

oxidising a chiral precursor prepared by the method of any of claims 10 to 14, to a 2,4,5-hydroxy-protected 2,4,5-trihydroxypentanoic acid or the corresponding pentanal,

converting the 1-hydroxy group of a chiral precursor prepared by the method of any one of claims 10 to 14, to a leaving group, substituting the leaving group with an azide to produce a 1,2,4-hydroxy-protected-5-azidopentane-1,2,4-triol, and reducing the azido group of the 1,2,4-hydroxy-protected 5-azidopentane-1,2,4-triol to an amine to produce a 1,2,4-hydroxy-protected 5-aminopentane-1,2,4-triol,

condensing the 2,4,5-hydroxy-protected 2,4,5-trihydroxypentanoic acid or corresponding pentanal and the 1,2,4-hydroxy-protected 5-aminopentane-1,2,4-triol to produce a hydroxy-protected N-(2′,4′,5′-trihydroxypentyl)-2,4,5-trihydroxypentanamide, or a hydroxy protected 6-azaundec-6-ene-1,2,4,8,10,11-hexaol

reducing the hydroxy-protected N-(2′,4′,5′-trihydroxypentyl)-2,4,5-trihydroxypentanamide 6-azaundec-6-ene-1,2,4,8,10,11-hexaol to the corresponding hydroxy-protected 6-aza-undecane 1,2,4,8,10,11-hexaol,

selectively deprotecting the 1 and 11 hydroxy groups to produce a 2,4,8,10 hydroxy-protected 6-aza-undecane-1,2,4,8,10,11-hexaol,

converting the 1 and 11 hydroxy groups to leaving groups, protecting the 6-aza nitrogen atom, and displacing the leaving groups with azide to produce an N-protected 2,4,8,10-hydroxy-protected 6-aza-1,11-diazidoundecane-2,4,8,10-tetraol,

deprotecting the 2,4,8 and 10 hydroxy groups to produce an N-protected 6-aza-1,11-diazido-undecane-2,4,8,10-tetraol,

reducing the azido groups of the N-protected 6-aza-1,11-diazido-undecane-2,4,8,11-tetraol to amino groups to produce an N-protected 1,11-diamino-6-aza-undecane-2,4,8,10-tetraol, and

removing the nitrogen protecting group to produce the desired stereoisomer of 1,11-diamino-6-aza-undecane-2,4,8,10-tetraol.

Protection of the 3,4-hydroxyl groups of the alkyl 3,4-dihydroxybutanoate ester will preferably be carried out by forming the acetonide using 2,2-dimethoxypropane. Selective protection of the primary hydroxyl group of the 1-(arylsulfinyl)-pentane-2,4,5-triol may be carried out using trityl chloride in the presence of a base such as dimethylaminopyridine (DMAP). Protection of the secondary 2,4-dihydroxy groups may then also be carried out by the formation of the acetonide. Reduction of the Pummerer rearranged product may be carried out by any suitable reducing agent such as lithium aluminium hydride. This C₅ reduction product is referred to, for convenience, as the “chiral precursor”.

Orthogonal protection of the chiral precursor obtained by selecting (2S)-malic acid as starting material, followed by selective deprotection of either the protected 1-hydroxyl group or the protected 5-hydroxyl group provides either the 2R,4S stereochemistry of the chiral precursor or the enantiomeric 2S,4R stereochemistry, respectively.

Conversion of this chiral precursor to the carboxylic acid and, separately, to the amine is a further important feature of the invention. These two steps provide the two halves of the 1,11-diamino-6-aza-undecane-2,4,8,10-tetraol product. Accordingly, a selection of the stereochemistry on the 2 and 4 positions of this chiral precursor will fix the stereochemistry on all four stereogenic centres of the product. So, for example, if the stereochemistry on carbons 2 and 4 of the chiral precursor are 2S and 4R, the stereochemistry on positions 8 and 10 of the undecane-2,4,8,10-tetraol will be 8R and 10S. Similarly, if the stereochemistry at positions 2 and 4 of the chiral precursor is 2R and 4S the stereochemistry at positions 8 and 10 of the product will be 8S and 10R.

The primary hydroxyl group of the chiral precursor may be converted to a leaving group by reaction with tosyl chloride. After substitution of the O-tosyl leaving group with sodium azide, the resulting azide group may be reduced to the amine with a reducing agent such as lithium aluminum hydride.

The oxidation of the primary hydroxyl group of the chiral precursor to the corresponding carboxylic and may be carried out with an oxidizing agent such as 2,2,6,6-tetramethylpiperidinyl-1-oxy, free radical, in the presence of sodium chlorite and sodium hypochlorite. Condensation of the amine and the carboxylic acid may be carried out in the presence of 1,1-carbonyl-diimidazole.

The reduction of the amide to the amine may be carried out with a reducing agent such as lithium aluminium hydride and cleavage of the primary O-trityl groups may be carried out using a reducing agent such as sodium in liquid ammonia. Conversion of the primary hydroxyl groups into leaving groups may be carried out by reaction with p-toluenesulfonyl chloride. This step also protects the nitrogen atom as the corresponding N-tosylate. Substitution of the O-tosyl leaving groups with azide may be carried out with a metal azide such as sodium azide and deprotection of the secondary hydroxyl groups may be carried out with p-toluenesulfonic acid in methanol. Conversion of the azido groups to amino groups may be carried out with a reducing agent such as palladium on carbon and removal of the N-tosylate group may be carried out with a reducing agent such as sodium in liquid ammonia.

In an embodiment of the invention, as shown in Schemes 3, 4 and 5, pavettamine was synthesised from (2S)-malic acid in a process designed to provide any one of the possible stereoisomers and which established the absolute configuration of the compound.

The starting material chosen was the four-carbon unit (2S)-malic acid, where stereochemistry at one position is already defined. Subsequent steps (Scheme 3) involved esterification to give 2, selective reduction of one of the esters to give 3 and acetonide protection to give 4. An additional carbon atom was introduced as a methyl sulfoxide 7 to give compound 8. The (R)-(+)-methyl p-tolylsulfoxide 7 was prepared from the anhydrous sodium salt of p-toluenesulfinic acid 5 via the menthyl ester 6. Reduction of the carbonyl was carried out using DIBALH, and in the presence of ZnBr₂ only the syn product 9 was formed. This reaction makes use of the chiral sulfoxide to control the stereoselectivity of the reduction: in the absence of ZnBr₂, only the anti product results. Removal of the acetonide followed by protection of the primary alcohol with the triphenylmethyl group gave 11. The syn diol was protected as an acetonide to give 12. In order to prepare the C₅ unit 14 (the “chiral precursor”) from compound 12, the sulfoxide group was transformed to a hydroxyl group by Pummerer rearrangement and reduction.

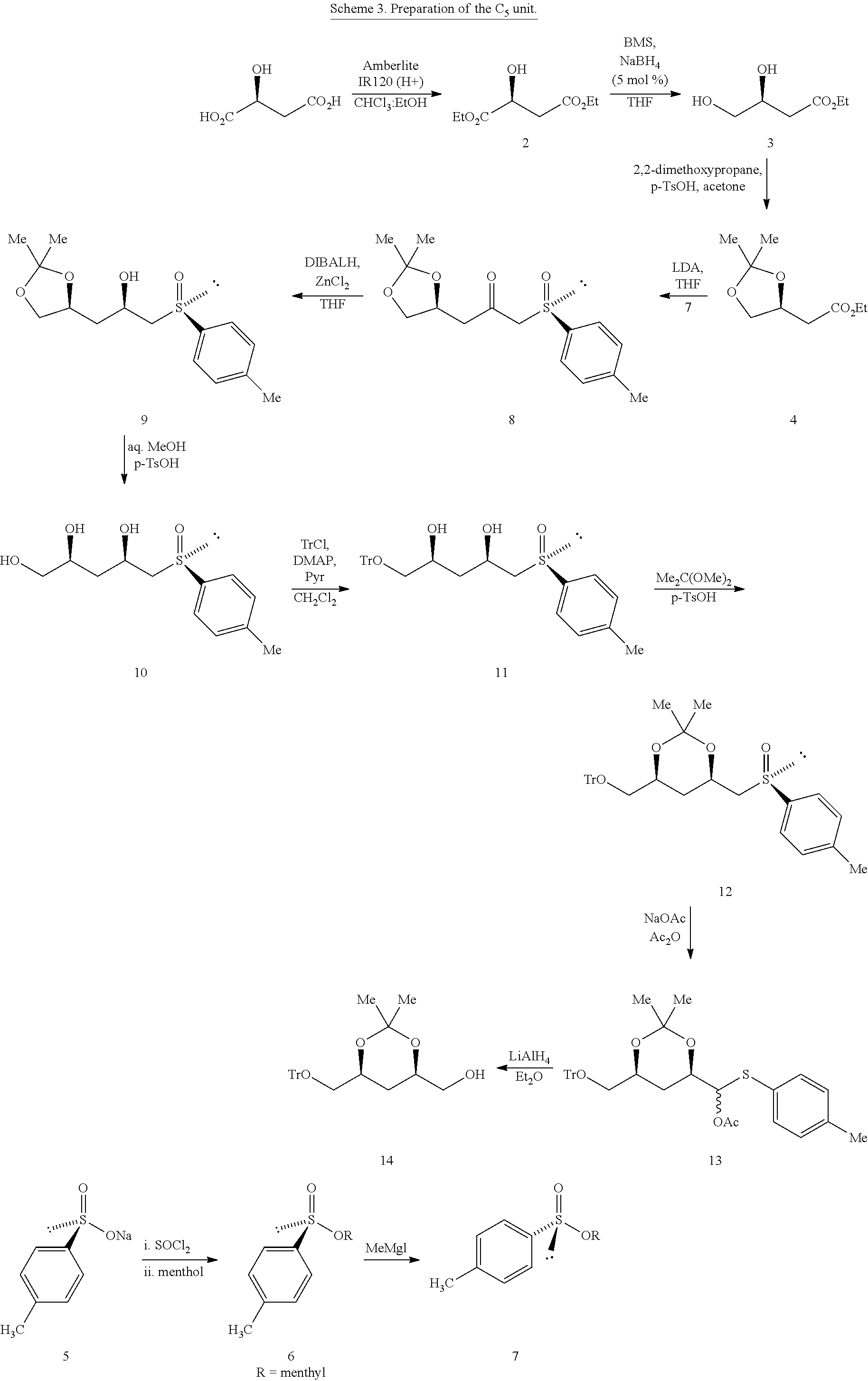

The C₅ unit was firstly functionalised to a carboxylic acid 15 by oxidation with TEMPO and NaOCl/NaClO₂ and, secondly, to an amine 18 via the tosylate 16 and azide 17 (Scheme 4).

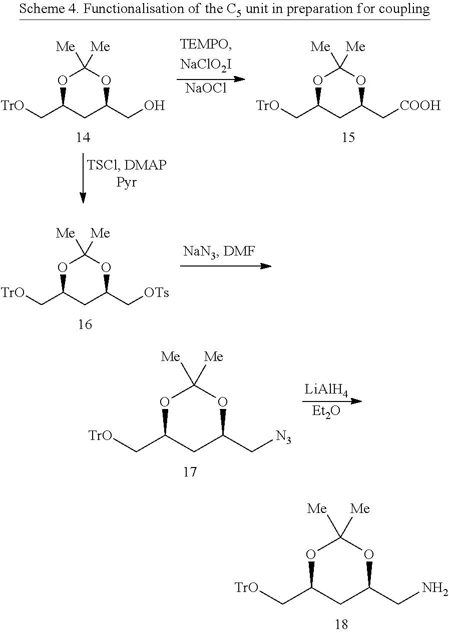

Preparation of the C₁₀ unit is shown in Scheme 5. The amine and carboxylic acid were linked to give amide 19 using the peptide coupling agent 1,1′-carbonyldiimidazole. Reduction of the amide to give an amine was achieved using LiAlH₄ in refluxing toluene. The triphenylmethyl deprotection was achieved using sodium in liquid ammonia to give compound 21.

Tosylation of compound 21 was carried out, followed by reaction with NaN₃ to give the diazide 23. Removal of the acetonide yielded diazide 24. Reduction of this compound under H₂ pressure (5 atm) using Pd/C as catalyst yielded amine 25. The final deprotection step was achieved using sodium in liquid ammonia. Clean-up of the final product was achieved using a Sephadex G10 column for salt removal, preceded by elution with water from a nitrile solid phase extraction column for removal of aromatic compounds.

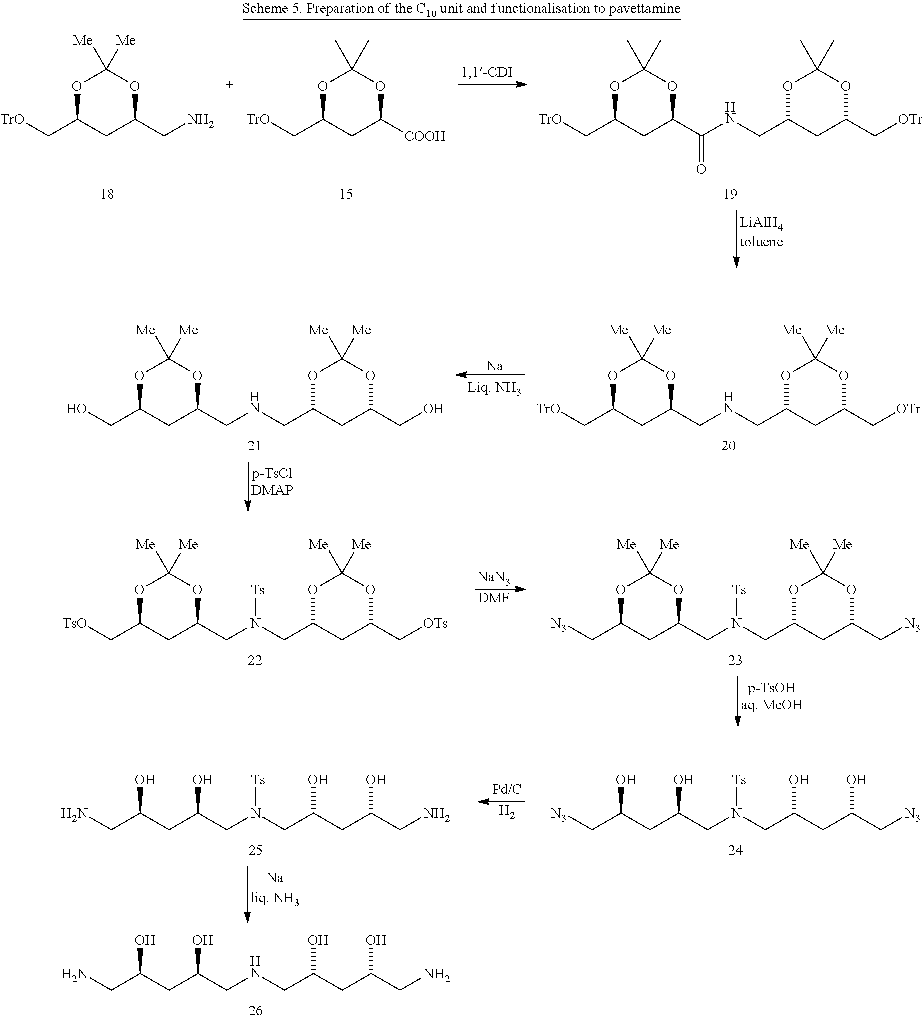

¹H and ¹³C NMR data of compound 26 proved to be identical to that of the natural product pavettamine. In addition, optical rotation measurements on compound 26 showed the sign of rotation to be minus, as found for the natural product. The synthesis thus showed that the absolute stereochemistry of the natural product pavettamine was identical to that of compound 26. Thin layer chromatography of pavettamine and compound 26 confirmed identical R_{f} values for both.

### Anticancer Activity

Results obtained when pavettamine was tested for activity against HeLa cancer cells showed that pavettamine was 10× more toxic to HeLa cancer cells (IC₅₀=0.2 μg/ml) than to human lymphocytes (IC₅₀=2 μg/ml).

In addition, when HeLa and MCF cells (breast cancer cells) were exposed to 200 μM pavetamine for 48 h, 30% cell death occurred for HeLa cells and 25% for the MCF cells. The MTT (3-(4,5-dimethyl-2-thiazolyl)-2,5-diphenyl-2H-tetrazolium bromide) assay (Sigma, St Louis, USA) was used to measure the cytotoxicity of pavettamine in these cell lines.

### Activity in Eukaryotic Versus Prokaryotic Cells

The algal (Selenastrum capricornumtum) growth inhibition test involves exposing the unicellular algae to the toxicant for 72 hrs, under defined conditions: at a temperature of 24° C. and in a test facility fitted with a cool white fluorescent light with a light intensity of ±4000 lux. The test facility is free of vapour, odours and dust which may be toxic to the algae.

Growth inhibition is measured as a reduction in growth rate relative to the control and is determined in terms of optical density. Definitive tests (testing serial dilutions) are carried out. The percentage growth inhibition in each test is usually reported as Effect or the 72 hr EC₂₀ or EC₅₀.

Results showed that pavettamine (1 mM to 0.063 mM) exhibited ≧98% growth inhibition at the concentrations used. These results suggest that pavettamine is a potent inhibitor of algal growth.

Pavettamine exhibited inhibitory activity towards the growth of fungal and yeast cells (for example Penicillium expansum, Aspergillus clavatus, Rhodotorula mucilaginosa and Debaryomyces hansenii) but showed no inhibitory activity towards the growth of bacterial cells (for example Staphylococcus intermedius (3 strains), Actinomyces pyogenes, Bordetella bronchiseptica, Pasteurella haemolytica and Sphingobacterium spritivorum). This selective inhibition of the growth of eukaryotes versus prokaryotes is a useful characteristic of the compound.

The invention accordingly extends to the use of 11-diamino-6-aza-undecane-2,4,8,10-tetraol, any one or more of its stereoisomers or any one or more of its salts in the preparation of a medicament for the treatment of cancer.

The invention further extends to a composition for the treatment of cancer, the composition comprising 11-diamino-6-aza-undecane-2,4,8,10-tetraol, any one or more of its stereoisomers or any one or more of its salts.

The invention further extends to a substance or composition for use in the treatment of cancer, the substance or composition comprising 11-diamino-6-aza-undecane-2,4,8,10-tetraol, any one or more of its stereoisomers or any one or more of its salts.

The invention further extends to a method of treating cancer, the method including the step of administering 11-diamino-6-aza-undecane-2,4,8,10-tetraol, its stereoisomers or its salts to a person or animal in need of treatment.

The stereoisomer is preferably (2S,4R,8R,10S)-1,11-diamino-6-aza-undecane-2,4,8,10-tetraol (1).

The invention extends further to a compound selected from the compounds 8, 9, 10, 11, 12, 13, 14, 15, 16, 17, 18, 19, 20, 21, 22, 23, 24 and 25 and their stereoisomers.

The invention is now described by way of example with reference to the following example and the figure in which

FIG. 1 shows the stereoisomers of pavettamine meeting the symmetry requirements of the molecule; and

FIG. 2 shows the stereoisomers of pavettamine lacking symmetry.

### The synthesis of natural pavettamine; (2S,4R,8R,10S)-1,11-Diamino-6-aza-undecane-2,4,8,10-tetraol (1)

### Diethyl (2S)-malate (2)

(2S)-Malic acid (100 g, 0.746 mol) was suspended in a mixture of CHCl₃/EtOH (3:4, 350 ml), Amberlite IR120 resin (H⁺ form, 40 g) was added and the mixture was heated under Dean-Stark reflux conditions. After reaction the resin beads were removed by filtration and washed with CHCl₃. The washings were added to the filtrate and the solvent was removed under reduced pressure. High vacuum distillation (115° C./1 mmHg) afforded diethyl malate (2) (132 g, 94%). [α]_{D} −10.6 (neat). ¹H NMR (300 MHz, CDCl₃): δ 4.449 (dd, 1H, J 4.7, 6.0, H-2), 4.248 (dq, 1H, J 10.9, 7.2, OCH₂CH₃), 4.229 (dq, 1H, J 10.9, 7.2, OCH₂CH₃), 4.200 (q, 2H, J 7.0, OCH₂CH₃), 3.52 (s, 1H, OH), 2.808 (dd, 1H, J 16.3, 4.7, H-2a), 2.751 (dd, 1H, J 16.3, 6.0, H-2b), 1.270 (t, 3H, J 7.0, Me), 1.235 (t, 3H, J 7.0, Me). ¹³C NMR (75 MHz, CDCl₃): δ 173.26S and 170.38S (C-1 and C-4), 67.25D (C-2), 61.85T and 60.83T (2×CH₂O), 38.69T (C-3), 14.00Q (2×CH₃).

### Ethyl (3S)-3,4-dihydroxybutanoate (3)

BH₃-DMS (251 mmol, 25.1 ml) was added dropwise over 30 min. to a stirred solution of diethyl (2S)-malate (2) (46.4 g, 0.244 mol) in dry THF (500 ml). After 35 min the solution was cooled in an ice-bath for 10 min. NaBH₄ (0.462 g, 5 mol %) was added and when the exothermic reaction subsided, the reaction was removed from the ice-bath and stirred at rt for an additional 40 min. The reaction was quenched by addition of EtOH (85 ml) and p-TsOH (2.32 g) and stirring at rt for 35 min. The mixture was then evaporated under reduced pressure on a rotary evaporator at 45° C. The resulting liquid was dissolved in benzene-EtOH (1:1, 500 mL) and concentrated. Benzene (400 ml) was added to the residue and concentrated again. This process was repeated twice more. The resulting oil was purified by column chromatography using EtOAc to afford ethyl (3S)-3,4-dihydroxybutanoate (3) (28.7 g, 79%). [α]_{D} −21.2 (c 2.3, CHCl₃). ¹H NMR (300 MHz, CDCl₃): δ 4.117 (q, J 7.0, OCH₂), 4.083 (dddd, 1H, J 8.0, 6.5, 4.9, 3.4, H-3), 3.780 (m, 1H, OH), 3.610 (dd, 1H, J 11.4, 3.4, H-4a), 3.464 (dd, 1H, J 11.4, 6.5, H-4b), 3.171 (s, 1H, OH), 2.481 (dd, 1H, J 16.3, 8.0, H-2a), 2.429 (dd, 1H, J 16.3, 4.9, H-2b), 1.220 (t, 3H, J 7.2, Me). ¹³C NMR (75 MHz, CDCl₃): δ 172.41S (C-1), 68.57D (C-3), 65.68T (C-4), 60.77T (CH₂O), 37.77T (C-2), 14.02Q (CH₃).

### Ethyl (3S)-3,4-O,O-isopropylidene-3,4-dihydroxybutanoate (4)

Ethyl (3S)-3,4-dihydroxybutanoate (3) (21.6 g, 0.146 mol) was dissolved in acetone (78 ml) and 2,2-dimethoxypropane (20 ml, 0.164 mol) and p-toluene-sulfonic acid (1.4 g, 7.4 mmol) were added. The reaction was allowed to stir for 30 min at rt and then neutralized by addition of Et₃N (3 ml). The solvent was removed and the residue was purified by column chromatography (EtOAc) to afford ethyl (3S)-3,4-O,O-isopropylidene-3,4-dihydroxybutanoate (4) (25.30 g, 92%). R_{f}=0.70 (EtOAc). [α]_{D} +19.2 (c 1.4, CHCl₃). ¹H NMR (300 MHz, CDCl₃): δ 4.373 (dddd, 1H, J 7.2, 6.2, 6.2, 5.9, H-3), 4.070 (q, 2H, J 7.2, OCH₂CH₃), 4.069 (dd, 1H, J 8.3, 5.9, H-4a), 3.568 (dd, 1H, J 8.3, 6.2, H-4b), 2.625 (dd, 1H, J 15.8, 6.2, H-2a), 2.426 (dd, 1H, J 15.8, 7.2, H-2b), 1.326 (s, 3H, (CH₃)₂C)), 1.269 (s, 3H, (CH₃)₂C)), 1.180 (t, 3H, J 7.2, OCH₂CH₃); ¹³C NMR (75 MHz, CDCl₃): δ 170.46S (C-1), 109.05S ((CH₃)₂C), 71.98D (C-3), 69.07T (C-4), 60.53T (CH₂O), 38.89T (C-2), 26.78Q and 25.42Q ((CH₃)₂C), 14.05Q (CH₃CH₂). HRMS (FAB): m/z 189.1127 (M+H)⁺; calcd for C₉H₁₇O₄: 189.1126.

### (1R,2S,5R)-(−)-Menthyl(S)-p-toluenesulfinate (6)

The powdered sodium salt of anhydrous p-toluenesulfinic acid (5) (80.0 g, 0.44 mol) was added in small portions to a solution of thionyl chloride (100 ml, 1.40 mol) in benzene (300 ml) at 0° C. The reaction was allowed to reach rt and the solvent was removed under reduced pressure. Excess thionyl chloride was removed by addition of benzene (200 ml) and evaporation under reduced pressure. The residue was diluted with anhydrous Et₂O (500 ml) (formation of a white precipitate of sodium chloride) and cooled at 0° C. A solution of (−)-menthol (69.4 g, 0.44 mol) in pyridine (70 ml) was added dropwise. After the addition was complete the mixture was stirred for 1 h at rt and hydrolysed with H₂O (200 ml). The organic layer was washed with 10% HCl (200 ml) and saturated brine (100 ml), dried over Na₂SO₄ and concentrated. The residue was diluted with acetone (200 ml), ˜5 drops 10M HCl were added, and allowed to crystallise at −20° C. After the filtration of the first crop of crystals, the mother liquor was concentrated to ˜50 ml, 1 drop 10M HCl was added and this was again allowed to crystallise at −20° C. This operation was repeated 3-4 times in total. Hexane was used to dilute the increasingly viscous mother liquor to improve crystallisation. The combined crops were finally recrystallised from hot acetone to give the pure (S)-sulfinate (6) as a white crystalline material (102.5 g, 78%). mp 108-109° C. [α]_{D}²¹ −201 (c 2.5, acetone).

### (R)-(+)-Methyl p-tolylsulfoxide (7)

A solution of methyl magnesium iodide [prepared from iodomethane (114 g, 803 mmol), and magnesium (16.0 g, 658 mmol)] in Et₂O (400 ml) was slowly added by cannula to a solution of (−)-(S)-menthyl-p-toluenesulfinate (6) (140 g, 475 mmol) in dry benzene (400 ml) between 0-10° C. After addition, the mixture was stirred at rt for 2 h and then hydrolysed with saturated aq. NH₄Cl solution (200 ml). The aqueous solution was extracted with Et₂O (2×400 ml). The organic layers were washed with saturated brine (200 ml), dried (Na₂SO₄) and concentrated in vacuo. The oily residue was mixed with hot hexane until formation of a light white cloudy precipitate and crystallization occurred overnight on cooling to −5° C. The solid material was recrystallised from Et₂O-hexane at −5° C. affording white crystals of (7) (57.4 g, 78%). mp 75-76° C. [α]_{D}²¹ +192 (c 4.0, CHCl₃); [α]_{D}²¹ +146 (c 2.0, acetone).

### (S(R),4S)-4,5-O,O-Isopropylidene-1-(p-tolylsulfinyl)-2-pentanone (8)

n-Butyllithium (1.5M in hexanes, 96.7 ml, 0.145 mol) was added to a solution of diisopropylamine (22.1 ml, 0.158 mol) in dry THF (160 ml) at −78° C. under argon. The mixture was stirred for 30 min at −78° C. and the solution was then allowed to reach −30° C. and (R)-(+)-methyl p-tolyl sulfoxide (7) (20.77 g, 0.135 mol) in dry THF (160 ml) was added. The solution went bright yellow at this stage. The mixture was stirred for 30 min while warming to 0° C., after which it was cooled to −40° C. and stirred for 5 min. Ethyl (3S)-3,4-O,O-isopropylidene-3,4-dihydroxy-butanoate (4) (12.37 g, 65.7 mmol) in dry THF (160 ml) was added slowly. On completion of addition the temperature was allowed to rise to rt and the reaction mixture was stirred for an additional 2 h. The reaction mixture was quenched by addition of saturated NH₄Cl solution and acidified with 1M HCl to pH 6. The mixture was extracted with EtOAc (3×100 ml), and the combined organic layers were washed with water and brine and dried over anhydrous Na₂SO₄. Removal of the solvent under reduced pressure gave viscous oil that was purified by column chromatography (hexane-EtOAc 1:9) to afford ketosulfoxide (8) (12.46 g, 64%). R_{f}=0.72 (hexane-EtOAc 1:9). [α]_{D} +148.9 (c 1.00, CHCl₃). ¹H NMR (300 MHz, CDCl₃): δ 7.48-7.24 (m, 4H, ArH), 4.333 (m, 1H, J 6.5, 6.3, 6.0, H-4), 4.037 (dd, 1H, J 8.3, 6.0, H-5b), 3.805 (s, 2H, H-1), 3.395 (dd, 1H, J 8.3, 6.5, H-5a), 2.888 (dd, 1H, J 17.1, 6.3, H-3b), 2.574 (dd, 1H, J 17.1, 6.5, H-3a), 2.355 (s, 3H, ArCH₃), 1.314 (s, 3H, (CH₃)₂C)), 1.256 (s, 3H, (CH₃)₂C). ¹³C NMR (75 MHz, CDCl₃): δ 199.27S (C-2), 141.93S, 139.17S, 129.85D and 123.78D (ArC), 108.73S ((CH₃)₂C), 70.90D (C-4), 68.80T (C-5), 67.68T (C-1), 48.82T (C-3), 26.52Q and 25.17Q ((CH₃)₂C), 21.14Q (ArCH₃). HRMS (FAB): m/z 297.1160 (M+H)⁺; calcd for C₁₅H₂₁SO₄: 297.1161.

### (S(R),2R,4S)-4,5-O,O-Isopropylidene-1-(p-tolylsulfinyl)-pentane-2,4,5-triol (9)

ZnCl₂ (8.24 g, 60.5 mmol) was flame-dried under vacuum in a 2-necked flask and cooled and dry THF (300 ml) was added. (S(R),4S)-4,5-O,O-isopropylidene-1-(p-tolylsulfinyl)-2-pentanone (8) (4.48 g, 15.1 mmol) in dry THF (100 ml) was added and this was allowed to stir at rt under argon for 2 h. The reaction mixture was cooled to −78° C. After stirring at −78° C. for 10 min, DIBALH (8.60 g, 10.8 ml, 60.5 mmol) was added slowly. The reaction was allowed to stir at low temperature for 1.5 h (TLC control) and then quenched by careful addition of saturated NH₄Cl solution at −78° C. The reaction was allowed to warm to rt and was extracted once with Et₂O. The organic solvent was removed under reduced pressure and the residue partitioned between water (pH 5) and EtOAc (3×50 ml). The organic solution was washed with brine, dried (Na₂SO₄) and evaporated to give a white solid. This material was purified by column chromatography (elution EtOAc) to afford the triol (9) (3.38 g, 75%) as a single diastereomer. Starting material (9%) was recovered. R_{f}=0.33 (EtOAc). [α]_{D} +130.0 (c 1.2, CHCl₃). ¹H NMR (300 MHz, CDCl₃): δ 7.55-7.29 (m, 4H, ArH), 4.317 (m, 1H, J 8.2, 7.8, 4.4, 3.9, 1.6, H-2), 4.252 (m, 1H, J 7.1, 7.0, 5.9, 4.9, H-4), 4.064 (dd, 1H, J 8.3, 5.9, H-5b), 3.922 (d, 1H, J 1.6, 2-OH), 3.583 (dd, 1H, J 8.3, 7.1, H-5a), 3.035 (dd, 1H, J 13.2, 7.8, H-1b), 2.822 (dd, 1H, J 13.2, 3.9, H-1a), 2.397 (s, 3H, ArCH₃), 1.878 (ddd, 1H, J 14.2, 8.2, 7.0, H-3b), 1.837 (ddd, 1H, J 14.2, 4.9, 4.4, H-3a), 1.386 (s, 3H, (CH₃)₂C), 1.318 (s, 3H, (CH₃)₂C); ¹³C NMR (75 MHz, CDCl₃): δ 141.76S, 140.36S, 130.01D and 124.02D (ArC), 109.27S ((CH₃)₂C), 73.96D (C-4), 69.34T (C-5), 66.74D (C-2), 62.97T (C-1), 39.89T (C-3), 26.79 Q and 25.63Q ((CH₃)₂C), 21.35Q (ArCH₃). HRMS (FAB): m/z 299.1317 (M+H)⁺; calcd for C₁₅H₂₃SO₄: 299.1317.

### (S(R),2R,4S)-1-(p-tolylsulfinyl)-pentane-2,4,5-triol (10)

(S(R),2R,4S)-4,5-O,O-Isopropylidene-1-(p-tolylsulfinyl)-pentane-2,4,5-triol (9) (5.05 g, 16.9 mmol) was dissolved in MeOH (150 ml) and water (40 ml) and p-toluenesulfonic acid (0.32 g, 10 mol %) was added. The reaction was heated under reflux for 1.5 h, after which TLC indicated that no starting material remained. Et₃N (1 ml) was added to neutralize the acid and the solvents were removed under reduced pressure. The residue was dissolved in water (60 ml) and extracted with EtOAc (50 ml) to remove any starting material. The aqueous layer was then continuously extracted with EtOAc for 2 d. The EtOAc solution was dried (Na₂SO₄) and evaporated to leave (S(R),2R,4S)-1-(p-tolylsulfinyl)-pentane-2,4,5-triol (10) (3.93 g, 90%) as an oil that solidified after drying under high vacuum. [α]_{D} +82.4 (c 1.0, MeOH). ¹H NMR (300 MHz, CDCl₃): δ 7.52-7.24 (m, 4H, ArH), 4.315 (m, 1H, H-2), 3.903 (m, 1H, H-4), 3.563 (dd, 1H, J 11.4, 3.6, H-5b), 3.454 (dd, 1H, J 11.4, 6.2, H-5a), 3.077 (dd, 1H, J 13.3, 7.4, H-1b), 2.819 (dd, 1H, J 13.3, 4.3, H-1a), 2.354 (s, 3H, ArCH₃), 1.768 (m, 1H, H-3b), 1.712 (m, 1H, H-3a); ¹³C NMR (75 MHz, CDCl₃): δ 141.94S, 139.90S, 130.10D and 124.18D (ArC), 71.07D (C-4), 67.16D (C-2), 66.33T (C-5), 62.62T (C-1), 39.13T (C-3), 21.35Q (ArCH₃). HRMS (FAB): m/z 259.1004 (M+H)⁺; calcd for C₁₂H₁₉SO₄: 259.1004.

### (S(R),2R,4S)-1-(p-Tolylsulfinyl)-5-(triphenylmethyloxy)pentane-2,4-diol (11)

4-Dimethylaminopyridine (0.36 g, 2.96 mmol) and triphenylmethyl chloride (4.64 g, 16.31 mmol) was added to a solution of (S(R),2R,4S)-1-(p-tolylsulfinyl)-pentane-2,4,5-triol (10) (3.83 g, 14.82 mmol) in CH₂Cl₂ (60 ml) and pyridine (4.8 ml, 59.3 mmol) and the reaction mixture stirred at rt for 2 d (TLC control). The reaction mixture was washed with 1M HCl (4×100 ml) and then with brine (100 ml). The organic layer was dried (Na₂SO₄) and evaporated to leave a yellow, viscous oil which was purified by column chromatography (elution hexane:EtOAc 1:4) to afford (S(R),2R,4S)-1-(p-tolylsulfinyl)-5-(triphenylmethyloxy)pentane-2,4-diol (11) (7.26 g, 98%). R_{f}=0.38 (hexane-EtOAc 1:4). [α]_{D} +89.3 (c 0.98, CHCl₃). ¹H NMR (300 MHz, CDCl₃): δ 7.61-7.18 (m, 19H, ArH), 4.34 (m, 2H, H-2 and OH), 4.016 (m, 1H, H-4), 3.26 (br s, 1H, OH), 3.095 (m, 2H, H-5), 3.017 (dd, 1H, J 13.2, 8.0, H-1b), 2.772 (dd, 1H, J 13.2, 3.6, H-1a), 2.394 (s, 3H, ArCH₃), 1.690 (dd, 2H, J 6.2, 6.2, H-3); ¹³C NMR (75 MHz, CDCl₃): δ 143.69S, 141.77S, 140.38S, 130.00D, 128.55D, 127.79D, 127.04D and 124.02D (ArC), 86.66S (Ph₃C), 70.41D (C-4), 68.10D (C-2), 67.40T (C-5), 63.04T (C-1), 39.37T (C-3), 21.34Q (ArCH₃). HRMS (FAB): m/z 501.2099 (M+H)⁺; calcd for O₃₁H₃₃SO₄: 501.2100.

### (S(R),2R,4S)-2,4-O,O-Isopropylidene-1-(p-tolylsulfinyl)-5-(triphenylmethyloxy)-pentane-2,4-diol (12)

p-Toluenesulfonic acid (25 mg) was added to a stirred solution of (S(R),2R,4S)-1-(p-tolylsulfinyl)-5-(triphenylmethyloxy)pentane-2,4-diol (11) (1.00 g, 1.997 mmol) in 2,2-dimethoxypropane (5 ml) and acetone (20 ml). Et₃N (1 ml) was added after 35 min and the solvent removed under reduced pressure. Column chromatography of the residue with hexane-EtOAc (1:1) as eluent afforded (S(R),2R,4S)-2,4-O,O-isopropylidene-1-(p-tolylsulfinyl)-5-triphenylmethyloxypentane-2,4-diol (12) (0.96 g, 89%). R_{f}=0.51 (hexane-EtOAc 1:1). [α]D+25.2 (c 1.08, CHCl₃); ¹H NMR (300 MHz, CDCl₃): δ 7.42-7.26 (m, 19H, ArH), 4.085 (m, 1H, H-2), 3.963 (m, 1H, H-4), 3.221 (dd, 1H, J 9.3, 5.2, H-5b), 3.140 (dd, 1H, J 13.2, 6.9, H-1b), 2.961 (dd, 1H, J 9.3, 6.0, H-5a), 2.752 (dd, 1H, J 13.2, 5.4, H-1a), 2.399 (s, 3H, ArCH₃), 1.736 (ddd, 1H, J 12.7, 2.3, 2.3, H-3), 1.370 (ddd, J 12.6, 12.6, 12.6, H-3), 1.325 (s, 3H, (CH₃)₂C), 1.293 (s, 3H, (CH₃)₂C); ¹³C NMR (75 MHz, CDCl₃): δ 143.91S, 141.62S, 140.26S, 129.78D, 128.68D, 127.73D, 126.95D and 124.41D (ArC), 98.87S ((CH₃)₂C), 86.52S (Ph₃C), 68.19D (C-4), 67.02T (C-5), 63.91D (C-2), 63.11T (C-1), 33.60T (C-3), 29.69Q ((CH₃)₂C), 21.36Q (ArCH₃), 19.60Q ((CH₃)₂C). HRMS (FAB): m/z 540.2335 (M⁺); calcd for C₃₄H₃₆SO₄: 540.2334.

### (1RS,2R,4S)-1-Acetoxy-2,4-O,O-isopropylidene-1-(p-tolylsulfanyl)-5-(triphenyl-methyloxy)-pentane-2,4-diol (13)

(S(R),2R,4S)-2,4-O,O-Isopropylidene-1-(p-tolylsulfinyl)-5-(triphenylmethyloxy)-pentane-2,4-diol (12) (0.40 g, 0.74 mmol) was dissolved in acetic anhydride (20 ml) and sodium acetate (0.43 g, 5.18 mmol) was added. The reaction was heated at 130-140° C. in an oil bath for 4.5 h (TLC control). The acetic anhydride was removed by repeated evaporation with toluene under reduced pressure. The residue was purified by column chromatography (elution hexane-EtOAc 4:1), to afford (1RS,2R,4S)-1-acetoxy-2,4-O,O-isopropylidene-1-(p-tolylsulfanyl)-5-(tri-phenylmethyloxy)pentane-2,4-diol (13) (0.36 g, 83%) as a mixture of diastereo-mers. R_{f}=0.36 (hexane-EtOAc 4:1). ¹H NMR (300 MHz, CDCl₃): δ 7.42-7.00 (m, 19H, ArH), 6.032 (d, J 5.7, H-1) and 5.992 (d, J 4.9, H-1), 4.14-3.94 (m, 2H, H-2 and H-4), 3.25 (m, 1H, H-5b), 3.00 (m, 1H, H-5a), 2.320 (s, 3H, ArCH₃), 2.053 (s, 3H, OAc), 1.399 (s, 6H, (CH₃)₂C); ¹³C NMR (75 MHz, CDCl₃): δ 169.77S and 169.61S (C═O), 144.00S, 138.52S, 138.42S, 134.16S, 133.72S, 129.80D, 129.69D, 128.72D, 128.31D, 127.74D and 126.96D (ArC), 99.15S ((CH₃)₂C), 86.50S (Ph₃C), 83.24D and 82.84D (C-1), 70.52D and 69.92D (C-2), 68.18D (C-4), 67.28T and 67.15T (C-5), 30.51T (C-3), 29.87Q and 29.77Q ((CH₃)₂C), 21.13Q (ArCH₃), 20.96Q (CH₃C═O), 19.58 ((CH₃)₂C). HRMS (FAB: m/z 582.2440 (M⁺); calcd for C₃₆H₃₈SO₅: 582.2440.

### (2R,4S)-2,4-O,O-Isopropylidene-5-(triphenylmethyloxy)pentane-1,2,4-triol (14)

(1RS,2R,4S)-1-Acetoxy-2,4-O,O-isopropylidene-1-(p-tolylsulfanyl)-5-(triphenyl-methyloxy)pentane-2,4-diol (13) (320 mg, 0.55 mmol) was dissolved in dry Et₂O (30 ml) and LiAlH₄ (44 mg, 1.10 mmol) was added. After 1.5 h (TLC control) 2M NaOH was added dropwise until a white precipitate formed. Anhydrous Na₂SO₄ was added and the mixture filtered. The solid white residue was extracted twice more with Et₂O (50 ml) and the combined Et₂O solution evaporated to give a residue that was purified by column chromatography with hexane-EtOAc (3:2), to afford the triol (14) (185 mg, 80%). R_{f}=0.29 (hexane-EtOAc 3:2). [α]_{D} −28.6 (c 0.76, CHCl₃). ¹H NMR (300 MHz, CDCl₃): δ 7.46-7.24 (m, 15H, ArH), 4.02 (m, 2H, H-2 and H-4), 3.602 (dd, 1H, J 11.4, 3.0, H-1b), 3.494 (dd, 1H, J 11.4, 6.3, H-1a), 3.259 (dd, 1H, J 9.2, 5.3, H-5b), 2.997 (dd, 1H, J 9.2, 6.1, H-5a), 2.06 (s, 1H, 1-OH), 1.546 (ddd, J 12.8, 2.6, 2.6, H-3b), 1.452 (s, 3H, (CH₃)₂C), 1.396 (s, 3H, (CH₃)₂C), 1.294 (ddd, 1H, J 12.0, 12.0, 12.0, H-3a); ¹³C NMR (75 MHz, CDCl₃): δ 144.00S, 128.70D, 128.43S, 127.73D and 126.93D (ArC), 98.67S ((CH₃)₂C), 86.48S (Ph₃C), 69.52D (C-2), 68.02D (C-4), 67.26T (C-5), 66.08T (C-1), 29.88Q ((CH₃)₂C), 29.81T (C-3), 19.84Q ((CH₃)₂C). HRMS (FAB): m/z 418.2144 (M⁺); calcd for C₂₇H₃₀O₄: 418.2144.

### (2R,4S)-2,4-O,O-Isopropylidene-5-(triphenylmethyloxy)pentanoic acid (15)

(2R,4S)-2,4-O,O-Isopropylidene-5-(triphenylmethyloxy)pentane-1,2,4-triol (14) (0.50 g, 1.19 mmol) was dissolved in acetonitrile (10 ml). To this solution were added TEMPO (13 mg, 0.08 mmol), sodium chlorite (269 mg, 2.38 mmol) in water (1 ml), buffer (7.5 ml of a 1:1 mixture of a 0.67M NaH₂PO₄ and a 0.67M Na₂HPO₄ solution) and bleach solution (89 μl of a 2% m/v solution, 0.024 mmol) in 0.5 mL water. The reaction was allowed to stir overnight at 35° C. Water (10 ml) was added and the reaction was cooled on ice prior to addition of sodium metabisulfite (400 mg). After 30 min the reaction was extracted with EtOAc (20 ml) and the organic layer washed with brine and dried (MgSO₄). The material was purified by column chromatography (elution CHCl₃-MeOH 4:1) to afford the carboxylic acid (15) (0.49 g, 95%). R_{f}=0.47 (CHCl₃-MeOH 4:1). ¹H NMR (300 MHz, CDCl₃): δ 7.45-7.24 (m, 15H, ArH), 4.595 (dd, 1H, J 12.3, 3.0, H-2), 4.153 (m, 1H, H-4), 3.361 (dd, 1-H, J 9.3, 5.2, H-5b), 3.138 (dd, 1H, J 9.3, 5.9, H-5a), 2.192 (ddd, 1H, J 13.2, 2.8, 2.6, H-3b), 1.48 (m, 1H, H-3a), 1.476 (s, 3H, (CH₃)₂C), 1.453 (s, 3H, (CH₃)₂C); ¹³C NMR (75 MHz, CDCl₃): δ 173.74S (C-1), 143.87S (ipso C), 128.74D, 127.87D and 127.10D (ArC), 99.79S ((CH₃)₂C), 86.67S (Ph₃C), 68.38D (C-4), 68.27D (C-2), 66.71T (C-5), 30.77T (C-3), 29.69Q ((CH₃)₂C), 19.63Q ((CH₃)₂C). HRMS (FAB): m/z 433.2015 (M+H)⁺; calcd for C₂₇H₂₉O₅: 433.2015.

### (2R,4S)-2,4-O,O-Isopropylidene-1-(p-toluenesulfonyloxy)-5-(triphenylmethyloxy)-pentane-2,4-diol (16)

(2R,4S)-2,4-O,O-Isopropylidene-5-(triphenylmethyloxy)pentane-1,2,4-triol (14) (1.0 g, 2.39 mmol) was dissolved in CH₂Cl₂ (30 ml) and 4-DMAP (1.3 eq., 0.38 g, 3.11 mmol) was added. The reaction was cooled to 0° C. in an ice bath and p-toluenesulfonyl chloride (1.25 eq., 0.57 g, 2.99 mmol) was added. The mixture was allowed to stir at rt for 1 day. Water (25 ml) was added and the mixture stirred for 30 min. The organic layer was separated, dried (Na₂SO₄) and the solvent removed under reduced pressure. The product, a white solid, was purified by column chromatography (hexane-EtOAc 4:1 as eluant) to afford (2R,4S)-2,4-O,O-isopropylidene-1-(p-toluenesulfonyloxy)-5-(triphenylmethyloxy)-pentane-2,4-diol (16) (1.13 g, 83%). R_{f}=0.31 (hexane-EtOAc 4:1). ¹H NMR (300 MHz, CDCl₃): δ 7.90-7.22 (m, 19H, ArH), 4.11 (m, 1H, H-4), 4.07 (m, 1H, H-2), 3.979 (dd, 1H, J 10.3, 5.7, H-1b), 3.928 (dd, 1H, J 10.3, 5.0, H-1a), 3.217 (dd, 1H, J 9.3, 5.2, H-5b), 2.948 (dd, 1H, J 9.3, 5.8, H-5a), 2.411 (s, 3H, ArCH₃), 1.594 (ddd, 1H, J 12.9, 2.6, 2.6, H-3b), 1.365 (s, 3H, (CH₃)₂C), 1.301 (s, 3H, (CH₃)₂C), 1.148 (ddd, 1H, J 12.9, 11.9, 11.9, H-3a); ¹³C NMR (75 MHz, CDCl₃): δ 144.73S, 143.91S and 133.02S (ipso C), 129.74D, 128.67D, 127.98D, 127.75D and 126.98D (ArC), 98.78S ((CH₃)₂C), 86.52S (Ph₃C), 72.37T (C-1), 67.85D (C-2), 67.05T (C-5), 66.86D (C-4), 30.18T (C-3), 29.64Q ((CH₃)₂C), 21.57Q (ArCH₃), 19.53Q ((CH₃)₂C). HRMS (FAB): m/z 572.2232 (M⁺); calcd for C₃₄H₃₆SO₆ 572.2233.

### (2S,4R)-5-Azido-2,4-O,O-isopropylidene-1-(triphenylmethyloxy)pentane-2,4-diol (17)

(2R,4S)-2,4-O,O-Isopropylidene-1-(p-toluenesulfonyloxy)-5-(triphenylmethyloxy)-pentane-2,4-diol (16) (0.96 g, 1.68 mmol) was dissolved in DMF (50 ml) and NaN₃ (2.5 eq., 0.27 g, 4.19 mmol) was added. The reaction was heated at 90° C. for 3.5 h. After cooling diethyl ether (250 ml) was added and the organic layer was washed once with saturated brine. This brine washing was extracted once with a fresh portion of diethyl ether (250 ml). The combined diethyl ether layers were washed with saturated brine (6×400 ml), dried (Na₂SO₄) and evaporated under reduced pressure to give (2R,4S)-1-azido-2,4-O,O-isopropylidene-5-(triphenylmethyloxy)pentane-2,4-diol (17) (0.74 g, 100%) as a yellowish solid. The product was not purified but used in the next reaction. R_{f}=0.56 (hexane-EtOAc 4:1)]. ¹H NMR (300 MHz, CDCl₃): δ 7.42-7.15 (m, 15H), 4.11-3.99 (m, 2H, H-2 and H-4), 3.284 (dd, 1H, J 9.3, 5.2, H-1a), 3.254 (dd, 1H, J 12.7, 6.5, H-5a), 3.180 (dd, 1H, J 12.7, 4.1, H-5b), 3.010 (dd, 1H, J 9.3, 6.0, H-1b), 1.605 (ddd, 1H, J 12.9, 2.6, 2.6, H-3a), 1.465 (s, 3H, (CH₃)₂C), 1.421 (s, 3H, (CH₃)₂C), 1.301 (ddd, 1H, J 12.9, 11.6, 11.6, H-3b); ¹³C NMR (75 MHz, CDCl₃): δ 143.97S (ipso C), 128.70D, 127.76D and 126.98D (ArC), 98.86S ((CH₃)₂C), 86.54S (Ph₃C), 68.48D (C-4), 68.12D (C-2), 67.16T (C-1), 55.18T (C-5), 31.32T (C-3), 29.66Q ((CH₃)₂C), 19.69Q ((CH₃)₂C). HRMS (FAB): m/z 443.2209 (M⁺); calcd for C₂₇H₂₉N₃O₃ 443.2209.

### (2S,4R)-5-Amino-2,4-O,O-isopropylidene-1-(triphenylmethyloxy)pentane-2,4-diol (18)

(2S,4R)-5-Azido-2,4-O,O-isopropylidene-1-(triphenylmethyloxy)pentane-2,4-diol (17) (0.65 g, 1.47 mmol) was dissolved in dry diethyl ether (40 ml) and LiAlH₄ (59 mg, 1.47 mmol) was added in one portion. The reaction was stirred at rt for 2 h. The reaction was stopped by dropwise addition of 2M NaOH to give a white precipitate. After addition of solid Na₂SO₄, the solids were collected by filtration and extracted twice more with diethyl ether (50 ml). The combined diethyl ether solutions were evaporated to give a white solid which was purified by column chromatography (elution CHCl₃-methanol 4:1) to afford the amine (18) (0.51 g, 84%). R_{f}=0.45 (CHCl₃-methanol 4:1); [α]_{D} −25.2 (c 1.34, CHCl₃). ¹H NMR (500 MHz, CDCl₃): δ 7.45-7.19 (m, 15H, ArH), 4.020 (m, 1H, H-2), 3.832 (m, 1H, H-4), 3.248 (dd, 1H, J 9.2, 5.3, H-1a), 2.965 (dd, 1H, J 9.2, 6.0, H-1b), 2.700 (dd, 1H, J 13.0, 4.2, H-5a), 2.674 (dd, 1H, J 13.0, 6.8, H-5b), 1.553 (ddd, 1H, J 12.7, 2.4, 2.4, H-3a), 1.439 (s, 3H, (CH₃)₂C), 1.382 (s, 3H, (CH₃)₂C), 1.205 (m, 1H, H-3b); ¹³C NMR (75 MHz, CDCl₃): δ 144.01S (ipso C), 128.69D, 127.70D and 126.91D (ArC), 98.56S ((CH₃)₂C), 86.46S (Ph₃C), 70.32D (C-4), 68.19D (C-2), 67.30T (C-1), 47.23T (C-5), 31.47T (C-3), 29.94Q ((CH₃)₂C), 19.85Q ((CH₃)₂C). HRMS (FAB): m/z 418.2382 (M+H)⁺; calcd for O₂₇H₃₂NO₃ 418.2382.

### (2R,4S)—N-{(2′R,4′S)-2,4-O,O-isopropylidene-5′-(triphenylmethyloxy)pentan-1′-yl}-2,4-O,O-isopropylidene-5-(triphenylmethyloxy)pentanamide (19)

(2R,4S)-2,4-O,O-Isopropylidene-5-(triphenylmethyloxy)pentanoic acid (15) (0.44 g, 1.01 mmol) was dissolved in dry DMF (8 ml) and 1,1′-carbonyldiimidazole (0.17 g, 1.06 mmol) was added. The reaction mixture was stirred at rt for 10 min. and then at 45° C. for min. After cooling, (2S,4R)-5-amino-2,4-O,O-isopropyl idene-1-(triphenylmethyloxy)pentane-2,4-diol (18) (0.42 g, 1.01 mmol) in dry DMF (2 ml) was added and the reaction was stirred at rt for 3 h. The reaction was diluted with diethyl ether (30 ml) and washed once with brine. This brine washing was extracted once with diethyl ether (30 ml). The combined diethyl ether solution was washed with brine (×4), dried (Na₂SO₄) and evaporated under reduced pressure. The residue was purified by column chromatography (elution hexane-EtOAc 3:2) to afford the amide (19) as a white solid (0.69 g, 81%). R_{f}=0.54 (hexane-EtOAc 3:2); [α]_{D} −22.6 (c 0.78, CHCl₃). ¹H NMR (300 MHz, CDCl₃): δ 7.45-7.20 (m, 30H, ArH), 6.903 (dd, 1H, J 6.8, 5.3, NH), 4.336 (dd, 1H, J 12.0, 2.8, H-2), 4.08-3.93 (m, 3H, H-2′, H-4, H-4′), 3.512 (ddd, 1H, J 13.6, 6.8, 3.3, H-1′a), 3.247 and 3.233 (each a dd, 1H, J 9.3, 5.3, H-5a and H-5′ a), 3.110 (ddd, 1H, J 13.5, 7.0, 5.3, H-1′b), 2.190 (ddd, 1H, J 13.2, 2.7, 2.7, H-3a), 1.596 (ddd, 1H, J 12.8, 2.6, 2.6, H-3′ a), 1.490, 1.427, 1.421, and 1.387 (each s, 3H, (2×(CH₃)₂C), 1.38-1.13 (m, 2H, H-3′b and H-3b); ¹³C NMR (75 MHz, CDCl₃): δ 171.53S (C-1), 143.99S and 143.89S (ipso C), 128.70D, 127.73D and 126.95D (ArC), 99.02S and 98.70S (2×(CH₃)₂C), 86.53S and 86.49S (2×Ph₃C), 69.45D (C-2), 68.61D and 68.16D (C-4 and C-4′), 67.93D (C-2′), 67.22T and 66.90T (C-5 and C-5′), 43.43T (C-1′), 31.69T (C-3), 31.27T (C-3′), 29.87Q and 29.73Q ((CH₃)₂C), 19.85Q and 19.66Q ((CH₃)₂C). HRMS (FAB): m/z 831.4135 (M⁺); calcd for C₅₄H₅₇NO₇ 831.4135.

### (2S,4R,8R,10S)-6-aza-2,4:8,10-di-O,O-isopropylidene-1,11-di(triphenylmethyl-oxy)-undecane-2,4,8,10-tetraol (20)

(2R,4S)—N-{(2′R,4′S)-2,4-O,O-Isopropyl idene-5′-triphenylmethyloxypentan-1′-yl}-2,4-O,O-isopropylidene-5-triphenylmethyloxypentanamide (19) (0.266 g, 0.32 mmol) was dissolved in dry toluene (7 ml). LiAlH₄ (72 mg) was added and the reaction refluxed for 2 h (TLC control). The reaction was quenched by addition of a few drops of water. After stirring for 15 min diethyl ether (30 ml) was added followed by solid anhydrous Na₂SO₄. The organic layer was filtered off and the solid material was extracted with diethyl ether (4×20 ml). The combined diethyl ether solutions gave a viscous oil that was purified by column chromatography (elution EtOAc-hexane 4:1) to give (2S,4R,8R,10S)-6-aza-2,4:8,10-di-O-isopropyl-idene-1,10-di(triphenylmethyloxy)-undecane-2,4,8,10-tetraol (20) (204 mg, 78%). R_{f}=0.35 (EtOAc-hexane 4:1). [α]_{D} −33.5 (c 0.85, CHCl₃). ¹H NMR (300 MHz, CDCl₃): δ 7.47-7.20 (m, 30H, ArH), 4.03 (m, 4H, H-4 and H-2), 3.257 (dd, 2H, J 9.3, 5.2, H-1a), 2.972 (dd, 2H, J 9.3, 5.9, H-1b), 2.687 (dd, 2H, J 12.2, 7.2, H-5a), 2.612 (dd, 2H, J 12.2, 4.1, H-1b), 1.593 (ddd, 2H, J 12.6, 2.3, 2.3, H-3a), 1.452 (s, 6H, (CH₃)₂C), 1.385 (s, 6H, (CH₃)₂C), 1.215 (ddd, 2H, J 12.6, 11.6, 11.6, H-3b); ¹³C NMR (75 MHz, CDCl₃): δ 144.05S (ipso C), 128.72D, 127.71D and 126.92D (ArC), 98.59S ((CH₃)₂C), 86.46S (Ph₃C), 68.31D (C-2), 68.00T (C-1), 67.37D (C-4), 54.90T (C-5), 32.17T (C-3), 29.99Q ((CH₃)₂C), 19.86Q ((CH₃)₂C). HRMS (FAB): m/z 818.4420 (M+H)⁺; calcd for C₅₄H₆₀NO₆ 818.4421.

### (2S,4R,8R,10S)-6-Aza-2,4:8,10-di-O-isopropylidene-undecane-1,2,4,8,10,11-hexaol (21)

(2S,4R,8R,10S)-1,10-Di(triphenyl methyloxy)-6-aza-2,4:8,10-di-O-isopropyl idene-undecane-2,4,8,10-tetraol (20) (0.286 g, 0.35 mmol) was dissolved in dry THF (12 ml) and liquid ammonia (25 ml, distilled from sodium) was added to the solution kept at −78° C. Sodium metal (20 eq.) was added in small pieces in four batches until a permanent blue colour was obtained. After 1 h a few drops of EtOH were added to the reaction, followed 5 min later by solid NH₄Cl (4 g). Ammonia was evaporated by gentle warming and the residue extracted with CH₂Cl₂ (20 ml). The CH₂Cl₂ was dried (Na₂SO₄) and evaporated. The product was purified by column chromatography (elution CHCl₃—MeOH 4:1) to afford the hexaol (21) (70 mg, 60%). R_{f}=0.46 (CHCl₃—MeOH 4:1). ¹H NMR (300 MHz, CDCl₃): δ 4.06 (m, 2H, H-4), 3.97 (m, 2H, H-2), 3.575 (dd, 2H, J 11.4, 3.4, H-1a), 3.477 (dd, 2H, J 11.4, 6.0, H-1b), 2.732 (dd, 2H, J 12.0, 8.0, H-5a), 2.630 (dd, 2H, J 12.0, 4.0, H-5b), 2.55 (br s, 2H, 1-OH), 1.443 (s, 6H, (CH₃)₂C), 1.384 (ddd, 2H, J 12.9, 3.1, 3.1, H-3a), 1.371 (s, 6H, (CH₃)₂C), 1.309 (ddd, 2H, J 12.8, 11.1, 11.1, H-3b); ¹³C NMR (75 MHz, CDCl₃) δ 98.92S ((CH₃)₂C), 69.39D (C-2), 67.37D (C-4), 65.93T (C-1), 54.52T (C-5), 30.07T (C-3), 29.94Q ((CH₃)₂C), 19.92Q ((CH₃)₂C). HRMS (FAB): m/z 334.2229 (M+H)⁺; calcd for C₁₆H₃₂NO₆ 334.2230.

### (2S,4R,8R,10S)-6-Aza-N-(p-toluenesulfonyl)-1,11-di-(p-toluenesulfonyloxy)-2,4:8,10-di-O,O-isopropylidene-undecane-2,4,8,10-tetraol (22)

(2S,4R,8R,10S)-6-Aza-2,4:8,10-di-O,O-isopropyl idene-undecane-1,2,4,8,10,11-hexaol (21) (58 mg, 0.17 mmol) was dissolved in CH₂Cl₂ (5 ml) and DMAP (128 mg, 1.05 mmol) and tosyl chloride (194 mg, 1.02 mmol) were added. The reaction was allowed to stir for 24 h at rt. The reaction mixture was partitioned between CH₂Cl₂ and water and the organic layer dried (Na₂SO₄) and evaporated. The product was purified by column chromatography (elution hexane-EtOAc 3:2 to hexane-EtOAc 1:1) to afford the product (22) (127 mg, 92%). R_{f}=0.42 (hexane-EtOAc 3:2). ¹H NMR (300 MHz, CDCl₃): δ 7.781 (d, 4H, J 8.4, ArH-3), 7.642 (d, 2H, J 8.4, ArH-3), 7.300 (d, 4H, J 8.4, ArH-2), 7.214 (d, 2H, J 8.4, ArH-2), 4.07-3.97 (m, 4H, H-2 and H-4), 3.928 (dd, 2H, J 10.1, 5.4, H-1a), 3.866 (dd, 2H, J 10.1, 4.7, H-1b), 3.278 (dd, 2H, J 14.8, 4.2, H-5a), 3.174 (dd, 2H, J 14.8, 7.2, H-5b), 2.419 (s, 6H, ArCH₃), 2.386 (s, 3H, ArCH₃), 1.446 (ddd, 2H, J 12.7, 2.3, 2.3, H-3a), 1.214 (s, 6H, (CH₃)₂C), 1.208 (s, 6H, (CH₃)₂C), 1.069 (ddd, 2H, J 12.7, 11.6, 11.6, H-3b); ¹³C NMR (75 MHz, CDCl₃) δ 144.82S, 143.38S, 137.29S and 132.90S (ipso C), 129.80D, 129.60D, 127.95D and 127.18D (ArC), 98.87S ((CH₃)₂C), 72.09T (C-1), 67.75D and 66.67D (C-2 and C-4), 54.15T (C-5), 30.06T (C-3), 29.63Q ((CH₃)₂C), 21.57Q (2×ArCH₃) and 21.39Q (ArCH₃), 19.38 ((CH₃)₂C). HRMS (FAB): m/z 795.2412 (M⁺); calcd for C₃₇H₄₉NS₃O₁₂ 795.2417.

### (2S,4R,8R,10S)-6-Aza-1,11-diazido-2,4:8,10-di-O-isopropylidene-N-(p-toluene-sulfonyl)-undecane-2,4,8,10-tetraol (23)

(2S,4R,8R,10S)-6-Aza-2,4:8,10-di-O,O-isopropylidene-N-(p-toluenesulfonyl)-1,11-di-(p-toluenesulfonyloxy)-undecane-2,4,8,10-tetraol (22) (117 mg, 0.15 mmol) was dissolved in DMF (6 ml) and sodium azide (48 mg, 0.74 mmol) was added and the reaction heated at 95° C. for 4 h. The reaction mixture was cooled, diluted with diethyl ether (50 ml) and washed with brine. The brine layer in turn was extracted once with diethyl ether (50 ml). The combined diethyl ether solutions were washed with brine (7×100 ml), dried (Na₂SO₄) and evaporated to afford the azide (23) (70 mg, 89%); ¹H NMR (300 MHz, CDCl₃): δ 7.681 (d, 2H, J 8.4, ArH-3), 7.267 (d, 2H, J 8.4, ArH-2), 4.110 (dddd, 2H, J 11.6, 7.1, 4.4, 2.7, H-4), 3.990 (dddd, 2H, J 11.6, 5.6, 4.4, 2.7, H-2), 3.339 (dd, 2H, J 14.7, 4.4, H-5a), 3.243 (dd, 2H, J 14.8, 7.1, H-5b), 3.208 (dd, 2H, J 13.0, 5.7, H-1a), 3.158 (dd, 2H, J 13.0, 4.4, H-1b), 2.394 (s, 3H, ArCH₃), 1.460 (ddd, 2H, J 12.8, 2.6, 2.6, H-3a), 1.331 (s, 6H, (CH₃)₂C), 1.308 (s, 6H, (CH₃)₂C), 1.223 (ddd, 2H, J 12.8, 11.6, 11.6, H-3b); ¹³C NMR (75 MHz, CDCl₃) δ 143.37S and 137.32S (ipso ArC), 129.60D and 127.24D (ArC), 98.97S ((CH₃)₂C), 68.32D (C-2), 68.07D (C-4), 55.03T (C-1), 54.40T (C-5), 31.13T (C-3), 29.82Q ((CH₃)₂C), 21.41Q (ArCH₃), 19.54Q ((CH₃)₂C). HRMS (FAB): m/z 538.24508 (M+H)⁺; calcd for C₂₃H₃₆N₇SO₆ 538.24472.

### (2S,4R,8R,10S)-6-Aza-1,11-diazido-N-(p-toluenesulfonyl)-undecane-2,4,8,10-tetraol (24)

(2S,4R,8R,10S)-6-Aza-1,11-diazido-2,4:8,10-di-O,O-isopropyl idene-N-(p-toluene-sulfonyl)-undecane-2,4,8,10-tetraol (23) (0.104 g, 0.193 mmol) was dissolved in MeOH (5 ml) and water (1.5 ml) was added. To this mixture was added p-toluenesulfonic acid (8 mg) and the reaction was stirred at rt for 3 days. The solvent was removed under reduced pressure and the residue was purified by chromatography (elution EtOAc-hexane 9:1) to afford the deprotected tetraol (24) (80 mg, 90%); R_{f}=0.45 (EtOAc-hexane 9:1). ¹H NMR (300 MHz, CDCl₃): δ 7.670 (d, 2H, J 8.2, ArH-3), 7.322 (d, 2H, J 8.2, ArH-2), 4.73 (br s, 2H, OH), 4.21 (m, 2H, H-4), 4.03 (m, 2H, H-2), 3.65 (br s, 2H, OH), 3.356 (dd, 2H, J 12.4, 4.4, H-1a), 3.269 (dd, J 12.4, 6.4, H-1b), 3.065 (dd, 2H, J 14.6, 7.8, H-5a), 3.037 (dd, 2H, J 14.6, 3.4, H-5b), 2.428 (s, 3H, ArCH₃), 1.06 (m, 4H, H-3); ¹³C NMR NMR (75 MHz, CDCl₃): δ 143.96S and 134.93S (ipso ArC), 129.89D and 127.39D (ArC), 70.91D and 70.61D (C-2 and C-4), 56.90T (C-1), 56.64T (C-5), 37.08T (C-3), 21.55Q (ArCH₃). HRMS (FAB): m/z 458.1822 (M+H)⁺; calcd for C₁₇H₂₈N₇SO₆ 458.1822.

### (2S,4R,8R,10S)-1,11-Diamino-6-aza-N-(p-toluenesulfonyl)-undecane-2,4,8,10-tetraol (25)

A solution of (2S,4R,8R,10S)-6-aza-1,11-diazido-N-(p-toluenesulfonyl)-undecane-2,4,8,10-tetraol (24) (129 mg, 0.281 mmol) in MeOH (5 ml) and 5% Pd/C (26 mg) in a small Parr reactor was stirred under H₂ at 5 atm at rt for 4 h. The reaction mixture was filtered to remove the catalyst and the solvent evaporated to afford the diamine (25) (0.116 mg, 100%) that was used without further purification. ¹³C NMR (75 MHz, CDCl₃): δ 143.55S and 135.17S (ipso ArC), 129.77D and 127.42D (ArC), 70.96D (C-2), 69.17D (C-4), 56.72T (C-5), 47.26T (C-1), 38.45T (C-3), 21.50Q (ArCH₃). HRMS (FAB): m/z 406.2012 (M+H)⁺; calcd for C₁₇H₃₂N₃SO₆ 406.2012.

### (2S,4R,8R,10S)-1,11-Diamino-6-aza-undecane-2,4,8,10-tetraol (synthetic pavettamine) (26)

(2S,4R,8R,10S)-1,11-Diamino-6-aza-N-(p-toluenesulfonyl)-undecane-2,4,8,10-tetraol (25) (63 mg, 0.155 mmol) was partially dissolved in dry dioxane (1 ml) and dry THF (15 ml) was added. Liquid ammonia (15 ml) was added and Na metal (40 mg) was added in three portions to give a blue solution. The reaction was allowed to stir at −78° C. for 1 h. A few drops of EtOH were added until the reaction turned colourless. The reaction mixture was removed from the cooling bath, the ammonia allowed to evaporate and 10M HCl (120 μl) added to the residue. The reaction mixture was filtered and the precipitate was dissolved in a small volume of distilled water and loaded on a Strata CN Phenomenex solid phase extraction column that had been prewashed with MeOH and then water. The sample was eluted with two column volumes of water, the solvent was removed under reduced pressure and two-thirds of the material was dissolved in a minimum amount of water before loading on a Sephadex G10 column (6 ml gel). The sample was eluted with distilled water. Fractions containing product eluted immediately before fractions containing salts. Combined fractions containing product were evaporated to give (2S,4R,8R,10S)-1,11-diamino-6-aza-undecane-2,4,8,10-tetraol (26) (11 mg, 40%). [α]_{D} −16.3 (c 0.49, H₂O); HRMS (FAB): m/z 251.18449 (M⁺); calcd for C₁₀H₂₅N₃O₄ 251.18451. ¹H and ¹³C NMR data identical to that of natural pavettamine, see Table 1.

¹H and ¹³C NMR data were identical to those of natural pavettamine and the sign of optical rotation was minus, as for pavettamine. (2S,4R,8R,10S)-1,11-Diamino-6-aza-undecane-2,4,8,10-tetraol (26) was identical to natural pavettamine (1), indicating that the absolute stereochemistry of pavettamine is that shown in structure (1).

1. A compound which is selected from the stereoisomers
2S,4R,8S,10R (A) and 2S,4S,6R,10R (B).
2R,4S,8S,10R (D)
2S,4S,8S,10S (E) and 2R,4R,8R,10R (F)
2R,4S,8R,10R (G) and 2S,4R,8S,10S(H)
2S,4R,8R,10R (I) and 2R,4S,8S,10S (J)
of 1,11-diamino-6-aza-undecane-2,4,8,10-tetraol.

2. (canceled)

3. A method of selectively preparing a stereoisomer of 1,11-diamino-6-aza-undecane-2,4,8,10-tetraol, the method including the steps of
providing a 2,4-hydroxy protected 1,5-difunctionalised-2,4-hydroxypentane in which
the stereochemistry on the 2 and 4 positions is selected from one of 2R,4R; 2R,4S; 2S,4R and 2S,4S,
the functional groups on the 1 and 5 carbon atoms are selected so that the 1-carbon and the 5-carbon can both, selectively, be converted to a carboxylic acid or an aldehyde, and so that the functional groups on the 1-carbon and the 5-carbon can both, selectively, be converted to an amine,
converting either the 1-carbon or the 5-carbon into a carboxylic acid or an aldehyde to produce a first intermediate,
providing the same 2,4-hydroxy protected 1,5-difunctionalised-2,4-hydroxypentane and converting the functional group on the 1-carbon or the 5-carbon to an amine to produce a second intermediate,
condensing the first and the second intermediates to form the corresponding amide or imine; and
reducing the amide or the imine to produce the stereoisomer of 2,4,8,10-hydroxy protected 1,11-difunctionalised-6-aza-undecane-2,4,8,10-tetraol.

4. A method as claimed in claim 3, in which the stereoisomer is selected from the stereoisomers
2S,4R,8S,10R (A) and 2S,4S,6R,10R (B).
2S,4R,8R,10S (C) and 2R,4S,8S,10R (D)
2S,4S,8S,10S (E) and 2R,4R,8R,10R (F)
2R,4S,8R,10R (G) and 2S,4R,8S,10S(H)
2S,4R,8R,10R (I) and 2R,4S,8S,10S (J).

5. A method as claimed in claim 4, in which the stereoisomer is (2S,4R,8R,10S)-1,11-diamino-6-aza-undecane-2,4,8,10-tetraol.

6. A method as claimed in claim 3, in which the functional group which is selected so that either the 1-carbon or the 5-carbon can be converted into an amine is a protected hydroxyl group.

7. A method as claimed in claim 6, in which the protected hydroxyl group is an O-tosyl group.

8. A method as claimed in claim 3, in which the functional group which is selected so that either the 1-carbon or the 5-carbon can be converted into a carboxylic acid or an aldehyde is a hydroxyl group.

9. A method as claimed in claim 2, in which the 2,4-hydroxy protected 1,5-difunctionalised-2,4-hydroxypentane is a chiral precursor selected from 2R,4S or 2S,4R or 2S,4S or 2R,4R-1,2,4-hydroxy-protected pentane-1,2,4,5-tetraol which is prepared by
providing a 3,4-hydroxy-protected 3,4-dihydroxybutanoate ester selected from 3,4-dihydroxybutanoate esters having 3S or 3R stereochemistry,
introducing an additional carbon atom as a methylene group into the selected 3,4-dihydroxy protected 3,4-dihydroxybutanoate ester using an (R)-(+)-methylarylsulfoxide to produce a 4,5-dihydroxy-protected 1-(arylsulfinyl)-4,5-dihydroxypentan-2-one,
reducing the 4,5-hydroxy-protected 1-(arylsulfinyl)-4,5-dihydroxypentan-2-one with an alkylaluminium hydride to produce a 4,5-hydroxy-protected 1-(arylsulfinyl)-pentane-2,4,5-triol, in which the 4S isomer, in the presence of a zinc halide, produces the 2R,4S isomer and, in the absence of a zinc halide, produces the 2S,4S isomer, and in which the 4R isomer, in the presence of a zinc halide, produces the 2S,4R isomer, and in the absence of a zinc halide, produces the 2R,4R isomer,
deprotecting the 4,5-hydroxy-protected 1-(arylsulfinyl)-pentane-2,4,5-triol to produce the corresponding 1-(arylsulfinyl)-pentane-2,4,5-triol,
selectively protecting the 5-hydroxy group of the 1-(arylsulfinyl)-pentane-2,4,5-triol to produce a 5-hydroxy-protected 1-(arylsulfinyl)-pentane-2,4,5-triol,
selectively protecting the 2,4-hydroxy groups of the 5-hydroxy-protected 1-(arylsulfinyl)-pentane-2,4,5-triol to produce a 2,4,5-hydroxy-protected 1-(arylsulfinyl)-pentane-2,4,5-triol,
converting the 2,4,5-hydroxy-protected 1-(arylsulfinyl)-pentane-2,4,5-triol to the corresponding 2,4,5-hydroxy-protected 1-acetoxy-1-arylthio-5-pentane-2,4,5-triol by Pummerer rearrangement, and
reducing the 2,4,5-hydroxy-protected 1-acetoxy-1-arylthio-5-pentane-2,4,5-triol to produce the 2R,4S or 2S,4R or the 2S,4S or 2R,4R 1,2,4-hydroxy-protected pentane-1,2,4,5-tetraol, chiral precursor.

10. A method as claimed in claim 9, in which the 3,4-hydroxy-protected 3,4-dihydroxybutanoate ester having 3S or 3R stereochemistry, is prepared from (2R)-malic acid or (2S)-malic acid by esterification to produce the diester followed by selective reduction.

11. A method as claimed in claim 10 in which the selective reduction is carried out using a borohydride in the presence of borane-methyl sulfide complex (BMS).

12. A method as claimed in claim 9, in which the (R)-(+)-methylarylsulfoxide is (R)-(+)-methyl p-tolylsulfoxide.

13. A method as claimed in claim 9, in which the alkyl aluminium hydride is diisobutyl aluminium hydride (DIBALH) and the zinc halide is selected from zinc chloride and zinc bromide.

14. A method as claimed in claim 9, which includes the steps of
oxidising the chiral precursor to a 2,4,5-hydroxy-protected 2,4,5-trihydroxypentanoic acid or the corresponding pentanal,
converting the 1-hydroxy group of the chiral precursor to a leaving group, substituting the leaving group with an azide to produce a 1,2,4-hydroxy-protected-5-azidopentane-1,2,4-triol, and reducing the azido group of the 1,2,4-hydroxy-protected 5-azidopentane-1,2,4-triol to an amine to produce a 1,2,4-hydroxy-protected 5-aminopentane-1,2,4-triol,
condensing the 2,4,5-hydroxy-protected 2,4,5-trihydroxypentanoic acid or corresponding pentanal and the 1,2,4-hydroxy-protected 5-aminopentane-1,2,4-triol to produce a hydroxy-protected N-(2′,4′,5′-trihydroxypentyl)-2,4,5-trihydroxypentanamide, or a hydroxy protected 6-azaundec-6-ene-1,2,4,8,10,11-hexaol
reducing the hydroxy-protected N-(2′,4′,5′-trihydroxypentyl)-2,4,5-trihydroxypentanamide or the 6-azaundec-6-ene-1,2,4,8,10,11-hexaol to the corresponding hydroxy-protected 6-aza-undecane 1,2,4,8,10,11-hexaol,
selectively deprotecting the 1 and 11 hydroxy groups to produce a 2,4,8,10 hydroxy-protected 6-aza-undecane-1,2,4,8,10,11-hexaol,
converting the 1 and 11 hydroxy groups to leaving groups, protecting the 6-aza nitrogen atom, and displacing the leaving groups with azide to produce an N-protected 2,4,8,10-hydroxy-protected 6-aza-1,11-diazidoundecane-2,4,8,10-tetraol,
deprotecting the 2,4,8 and 10 hydroxy groups to produce an N-protected 6-aza-1,11-diazido-undecane-2,4,8,10-tetraol,
reducing the azido groups of the N-protected 6-aza-1,11-diazido-undecane-2,4,8,11-tetraol to amino groups to produce an N-protected 1,11-diamino-6-aza-undecane-2,4,8,10-tetraol, and
removing the nitrogen protecting group to produce the desired stereoisomer of 1,11-diamino-6-aza-undecane-2,4,8,10-tetraol.

15. A method as claimed in claim 14, in which oxidising the chiral precursor to the 2,4,5-hydroxy-protected 2,4,5-trihydroxypentanoic acid is carried out using 2,2,6,6-tetramethylpiperidinyl-1-oxy free radical, in the presence of sodium chlorite and sodium hypochlorite.

16. A method as claimed in claim 14, in which condensing the 2,4,5-hydroxy-protected 2,4,5-trihydroxypentanoic acid and the 1,2,4-hydroxy-protected 5-aminopentane-1,2,4-triol is carried out in the presence of 1,1-carbonyl-diimidazole.

17. A method of treating cancer, which method includes the step of administering any one or more of the stereoisomers of claim 1, or any one or more of the salts thereof to a person or animal in need of treatment.

18.-20. (canceled)

21. A compound selected from the compounds 19, 20, 21, 22, 23, 24 and 25 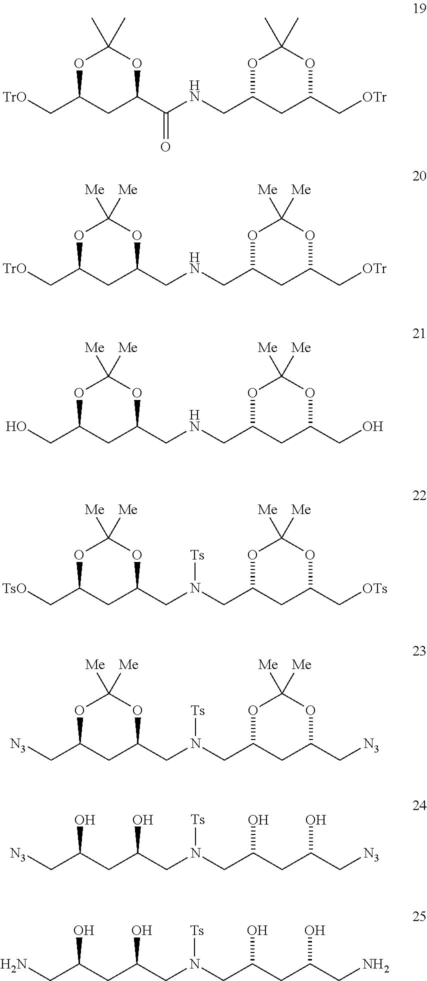
and any one of their stereoisomers.

22. A method as claimed in claim 15, in which condensing the 2,4,5-hydroxy-protected 2,4,5-trihydroxypentanoic acid and the 1,2,4-hydroxy-protected 5-aminopentane-1,2,4-triol is carried out in the presence of 1,1-carbonyl-diimidazole.
